(19) United States
(12) Patent Application Publication
(10) Pub. No.: US 2025/0256014 A1
(43) Pub. Date: Aug. 14, 2025

(54) ERGONOMIC FORWARD LEANING BREAST PUMPING CHAIR SYSTEMS AND DEVICES THEREOF

(71) Applicant: Restful Pump, Inc., Pembroke, MA (US)

(72) Inventor: Micolene BODDIE, Marshfield, MA (US)

(21) Appl. No.: 19/034,461

(22) Filed: Jan. 22, 2025

Related U.S. Application Data
(60) Provisional application No. 63/623,649, filed on Jan. 22, 2024.
(60) Provisional application No. 63/646,346, filed on May 13, 2024.
(60) Provisional application No. 63/691,144, filed on Sep. 5, 2024.

(51) Int. Cl. A61M 1/06; A47C 9/00; A61H 19/00; A61H 23/00

(52) U.S. Cl. CPC A61M 1/067; A61H 19/32; A61H 23/00; A61M 1/0697; A47C 9/005; A61H 2201/0149; A61H 2201/0192; A61H 2201/0207; A61H 2201/10; A61H 2201/1604; A61H 2201/1619; A61H 2203/0425; A61H 2205/082; A61M 2205/05; A61M 2205/36; A61M 2209/084

(57) ABSTRACT

Breast pumping chair systems are disclosed that include a chair assembly including a seat frame coupled to a base frame, feet coupled to opposing ends of the base frame, a seat support coupled to the seat frame, and an angle adjustment mechanism. The systems further include a breast pumping assembly including a chest frame coupled to the seat frame, a chest support coupled to the chest frame, a chest cushion angled to engage a sternum of a user in a forward leaning position, and a headrest post removably attached to the chest frame. The breast pumping assembly includes a milk collection system including bottle holders rotatably coupled to opposing sides of the chest frame. The systems also include a height adjustment mechanism to alter a height of the breast pumping assembly and a table rotatably coupled to the chair assembly and/or the breast pumping assembly via a table hinge.

### CROSS-REFERENCE TO RELATED APPLICATIONS

This application claims priority to U.S. Provisional Patent Application Ser. No. 63/691,144, filed Sep. 5, 2024, U.S. Provisional Patent Application Ser. No. 63/646,346, filed May 13, 2024, and U.S. Provisional Patent Application Ser. No. 63/623,649, filed Jan. 22, 2024, each of which is hereby incorporated by reference herein in its entirety.

### FIELD

This technology generally relates to breast pumping systems and, more particularly, to ergonomic forward leaning breast pumping chair systems that facilitate improved positioning and reduced medical complications for breast pumping women.

### BACKGROUND

Feeding infant children using breastmilk has several advantages including increased infant health, increased protection against infections, diseases, and viruses, improved digestion relative to other forms of nutrition, and healthier weight as infants grow. To build a store of milk for feeding an infant, mothers often pump breastmilk for later consumption. Mothers that choose to pump breastmilk may enjoy several benefits. For example, pumping after breastfeeding sessions may increase milk supply and/or provide nipple stimulation to increase and maintain milk supply.

Additionally, breast pumping provides convenience for mothers and family members by facilitating subsequent bottle feeding by family members that can thereby improve their bond with the baby. Further, mothers can return to the workplace, perform tasks, and/or rest, for example, while other caregivers bottle feed. Even further, some mothers choose to sell breastmilk, or donate extra breastmilk to a milk bank, and surrogates may breast pump and donate breastmilk after a baby is born and living with their parents.

Pumping breastmilk may also be medically beneficial or even necessary in some instances. For example, mothers may pump breastmilk for infants that have generally poor latch, suck difficulties, or other medical issues. Other mild to severe medical complications that may require a mother to pump breastmilk include infant prematurity, weakness, low muscle tone, ankyloglossia, necrotizing enterocolitis, (NEC) or other illnesses, disabilities, or difficulties. Pumping breastmilk is advantageous for the nursing mother if she has flat or inverted nipples or cannot nurse her infant due to post-surgical complications, mild to severe postpartum diagnoses or conditions, other life-threatening issues, or simply has an upper extremity disability (e.g., an amputee or another shoulder, elbow, or wrist diagnosis), back pain or other orthopedic diagnosis or co-morbidity.

However, many mothers struggle with milk production via breast pumping for many reasons, including stress and anxiety of caring for an infant, return to work, discomfort caused by breast pumping, and discomfort from the various body positions that must be maintained for extended periods of time during a breast pumping session. Additionally, nursing or pump bras introduce compression that may contribute to breast tissue complications, and/or obstructions that limit breast tissue massage, and may increase stress. Often, during a breast pumping session, a woman is seated with poor posture, leading to inefficient pumping, less milk extraction, and/or reduced milk production.

In addition to reduced milk expression, sub-optimal positioning during breast pumping can also lead to significant medical issues for women including neck, shoulder, and/or back pain or discomfort or other similar diagnoses, headaches or migraines, or exhaustion. Mothers that use current breast pumping devices in sub-optimal environments and positions may also have an increased susceptibility of developing clogged ducts. Current breast pumping devices also require mothers to hold bottles coupled to flanges, eliminating the ability to perform self-guided, therapeutic breast massage during breast pumping sessions to ensure efficient milk expression of the breasts and reducing the risk of medical complications of the breast tissue.

While the negative medical implications of current breast pumping systems and environments are significant, women that currently choose to breast pump also cannot productively rest or multitask, thereby increasing stress. Thus, current breast pumping systems and devices unfortunately have significant drawbacks.

### BRIEF DESCRIPTION OF THE DRAWINGS

FIG. 1 is a perspective view of an exemplary breast pumping chair system;

FIG. 2 is a perspective view of an exemplary chair assembly of the breast pumping chair system of FIG. 1;

FIG. 3 is a rear perspective view of an exemplary breast pumping assembly of the breast pumping chair system of FIG. 1;

FIG. 4 is a front perspective view of an exemplary breast pumping assembly of the breast pumping chair system of FIG. 1;

FIG. 5 is a perspective view of an exemplary bottle adjustment system of the breast pumping assembly of FIGS. 3-4;

FIG. 6 is a perspective view of another exemplary breast pumping chair system;

FIG. 7 is an exploded view of the breast pumping chair system of FIG. 6;

FIG. 8 is a side plan view of the breast pumping chair system of FIG. 6 in two positions facilitated by the base frame;

FIG. 9 is a perspective view of the breast pumping chair system of FIG. 6 with exemplary bottle attachment devices;

FIGS. 10A-B are perspective views of another exemplary breast pumping assembly of the breast pumping chair system of FIG. 9 with angle and height adjustment mechanisms;

FIG. 11 is a perspective view of the table adjustment mechanism 906 of the breast pumping chair system 600 of FIG. 9.

FIG. 12 is a perspective view of the breast pumping chair system of FIG. 9 with the table in a collapsed position;

FIG. 13 is a perspective view of an exemplary milk collection system with two ball joints coupled to a clamp attached to a collection system post;

FIG. 14 is a perspective view of the exemplary milk collection system of FIG. 12 with the clamp coupled to a table;

FIG. 15 is a side plan view of the milk collection system of FIG. 12 with the clamp disposed proximate a slider attachment of the milk collection system, which is coupled to a grab bar;

FIG. 16 is a side plan view of the milk collection system of FIG. 14 illustrating the attachment of the slider attachment;

FIG. 17 is a perspective view of another exemplary bottle holder;

FIG. 18 is a perspective view of the breast pumping chair system of FIGS. 9 and 11 with another exemplary milk collection system including another exemplary bottle holder;

FIG. 19 is a perspective view of the milk collection system of FIG. 18 attached to the grab bar;

FIGS. 20A-B are rear and front perspective views, respectively, of the bottle holder of the milk collection system of FIG. 18;

FIG. 21 is a perspective view of the milk collection system of FIG. 18 coupled to the collection system post;

FIG. 22 is a perspective view of another exemplary milk collection system with a ball joint housing;

FIGS. 23A-C are perspective views of another exemplary milk collection system with gears;

FIGS. 24A-B are perspective and plan views of an exemplary handle detached from and attached to, respectively, the grab bar;

FIGS. 25A-D illustrate an exemplary seatbelt system;

FIGS. 26A-B illustrate an exemplary tissue warming system; and

FIG. 27 illustrates a portable breast pumping positioning system.

### DETAILED DESCRIPTION

The disclosed technology generally relates to supportive, ergonomic breast pumping chair systems, breast pumping assemblies, and bottle adjustment systems that advantageously allow users to productively pump breastmilk while they restore their bodies, care for their physical and mental health, and decrease the risk of breast tissue complications and use as an ergonomic workstation, with or without the bottle holders. The systems, assemblies, and devices of the disclosed technology facilitate pressure avoidance on the lower pelvic region for a user (e.g., a user following a postpartum c-section or having diastasis recti or other medical complications) while plush and adjustable components lock into place for each unique user to ensure personalized, improved suction with each breast pump session. Supportive ergonomics of the exemplary breast pumping chair systems promote relaxation while facilitating gravity and provide comfort while breast pumping to reduce pain and exhaustion, and to extend breast pumping sessions to help mothers reach their breast milk production goals.

The breast pumping chair systems of this technology include a chair assembly and a breast pumping assembly that collectively provide multiple forward leaning positions for a user. For example, the breast pump chair systems can allow a user to be positioned 30 degrees into a forward leaning position to work, read, use a portable electronic device (e.g., a laptop), or multitask on a table coupled to the breast pumping assembly while breast pumping hands-free. Hands-free breast pumping allows users to perform self-breast tissue massage for greater breastmilk output and less susceptibility for clogged milk ducts. This position may also be used with or without the headrest. In another example, a position at 45 degrees (forward leaning) from prone is provided with a headrest coupled to the breast pumping assembly of the breast pumping chair system to allow a user to rest or sleep in a more forward leaning position for dangle breast pumping. Leveraging gravity while leaning forward ensures efficient milk expression and may reduce the risk of breast tissue complications, such as clogged milk ducts, breast abscess, mastitis, or other breast infections. Any forward learning position may be achieved with the disclosed breast pumping chair systems.

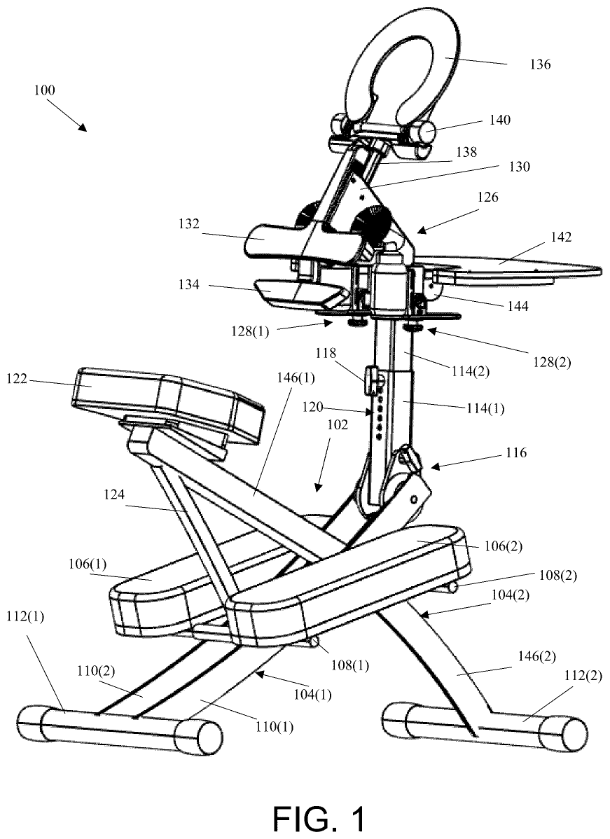

Referring now to FIG. 1, a perspective view of an exemplary breast pumping chair system 100 is illustrated. The breast pumping chair system 100 in this example includes a chair assembly 102 that includes first and second base supports 104(1) and 104(2) and first and second leg supports 106(1) and 106(2) coupled to the first and second base supports 104(1) and 104(2) although other arrangements of those components can also be provided. The first and second leg supports 106(1) and 104(2) can be cushioned and located on opposing sides of the second base support 104(2). Optionally, first and second cross-braces 108(1) and 108(2), respectively, can be attached to the second base support 104(2) and the first base support 104(1) although the first and second leg supports 106(1) and 106(2) can be attached in other ways in other examples. The first and second leg supports 106(1) and 104(2) and/or the first and second cross-braces 108(1) and 108(2), can be removed to allow a user to stand or sit, for example.

The first base support 104(1) in this example includes opposing first and second rails 110(1) and 110(2) coupled at one end to a first foot 112(1) extending substantially perpendicular to the first and second rails 110(1) and 110(2), respectively. However, the first base support 104(1), or one or more portions thereof, can be a tubular or monolithic structure in other examples. In this example in which the first base support 104(1) includes the opposing first and second rails 110(1) and 110(2) the chair assembly 102 includes a first support post 114(1) disposed between the first and second rails 110(1) and 110(2) toward another end of the first and second rails 110(1) and 110(2), respectively. The first support post 114(1) is rotatably coupled to the first base support 104(1) via a post locking mechanism 116.

In some examples, the post locking mechanism 116 allows rotation of the first support post 114(1) into at least two locked positions that facilitate positioning of a user at 30 degrees forward leaning and at 45 degrees from prone. However, in other examples, any number of forward leaning angled positions can be facilitated by the post locking mechanism 116 based on a rotation or other adjustment to the first support post 114(1) or other means. The post locking mechanism 116 can include a first pin 118 and through holes 120 on the first support post 114(1), for example, although other methods for rotating and/or retaining the first support post 114(1) in place at a desired angle of rotation relative to the first base support 104(1) can also be used in other examples.

The chair assembly 102 in this example further includes a seat 122, optionally cushioned, coupled to the second base support 104(2). The second base support 104(2) is coupled at one end to the seat 122 and at another end to a second foot 112(2) extending substantially perpendicular to the second base support 104(2). The second foot 112(2) can be substantially the same as, or different than, the first foot 112(1). One or more of the first or second feet 112(1) and 112(2) (e.g., only the second foot 112(2)) can include one or more wheels or casters to facilitate portability of the breast pumping chair system 100.

The breast pumping chair system 100 further includes a seat support 124 coupled between the first and second base supports 104(1) and 104(2) via which a height of the seat 122 can be locked and/or adjustable. In this example, the seat support 124 is coupled at one end to the second base support 104(2) proximate the seat 122, optionally via a hinged coupling that allows for movement of another end of the seat support 124 toward the second base support 104(2) and/or between the first and second rails 110(1) and 110(2), respectively. In other examples, the seat height is fixed and/or locked in place by engagement of the second end of the seat support 124 with the cross bracing of the first and second leg supports 106(1) and 106(2) and/or a portion of the first base support 104(1), and the seat 122 can be configured and/or supported in other ways in other examples. The breast pumping chair system 100 may also be disassembled (e.g., as a flat pack) for travel.

The breast pumping chair system 100 further includes the breast pumping assembly 126 that is configured to attach to, or be integral with, the chair assembly 102. More specifically, in this example, the breast pumping assembly 126 includes a second support post 114(2) that is configured to attach to the first support post 114(1) of the chair assembly 102 at one of a plurality of positions along a length of the first support post 114(1). The first support post 114(1) is sized to receive the second support post 114(2) in this example, although the opposite configuration can also be used in other examples along with other types of connections between the first and second support posts 114(1) and 114(2), respectively.

The positions along the length of the first support post 114(1) may correspond to a height of the breast pumping assembly 126, which is therefore adjustable to a height desired by a user. The attachment of the first support post 114(1) to the second support post 114(2) can be by way of first pin 118 and through holes 120 aligned on both the first and second support posts 114(1) and 114(2) although other methods for attaching the first and second support posts 114(1) and 114(2) and/or adjusting the height of the breast pumping assembly 126 can also be used.

The breast pumping assembly 126 in this example includes first and second bottle slider devices 128(1) and 128(2) which are rotatably and/or removably coupled to opposing sides of the second support post 114(2), as explained in more detail below. The first and second bottle slider devices 128(1) and 128(2) are adjustable in multiple dimensions for alignment in a comfortable position with respect to the breasts of a user. Additionally, the first and second bottle slider devices 128(1) and 128(2) are generally configured to hold a bottle or other breastmilk container or receptacle capable of receiving and retaining breastmilk expressed by a user of the breast pumping assembly 126, as also explained in more detail below.

The breast pumping assembly 126 may also include a rest support 130 coupled to the second support post 114(2) and including a sternum support 132 angled to engage a sternum of the user in a forward leaning position and an optional abdomen support 134 coupled to the rest support 130 below the sternum support 132. One or both of the sternum support 132 or abdomen support 134 can be cushioned. Additionally, the sternum support 132 may be adjustable longitudinally with respect to the rest support 130, such as within a track of the rest support 130 with lockable positions for the sternum support 132, for example. Also optionally, the abdomen support 134 may be adjustable longitudinally toward or away from the sternum support 132 or the rest support 130, for example, and other adjustments in other dimensions or directions for the sternum support 132 and/or abdomen support 134 may or may not be used in other examples.

The breast pumping assembly 126 also includes an optional headrest 136 that includes a headrest post 138, which may engage with the rest support 130 and thereby couple the headrest 136 to the rest support 130. Optionally, the headrest post 138 can be received by an aperture in the rest support 130 at any number of lockable positions along a length of the headrest post 138 to thereby facilitate locking the headrest 136 at a desired height or depth for a user. The headrest 136 can also be removable via the headrest post 138 from the rest support 130, if desired by a user. The headrest post 138 is coupled to an adjustable headrest support 140 to which the headrest 136, optionally cushioned, is attached. In this example, the headrest 136 is coupled to the headrest support 140 such that the headrest 136 is rotatable relative to the headrest support 140 and, optionally, adjustable toward and away from a user, although other types of headrests and/or methods of attaching and/or adjusting the headrest 136 can also be used in other examples.

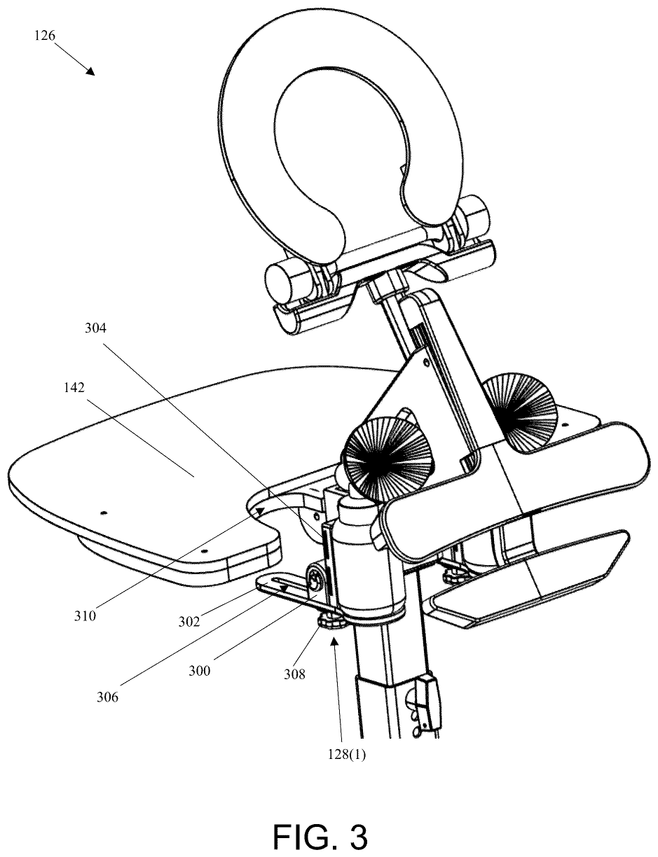

The breast pumping assembly 126 also includes a table 142 in this example, which is substantially perpendicular to the second support post 114(2), and/or substantially parallel to a floor or other flat surface on which the breast pumping chair system 100 is disposed, when in use. In some examples, the table 142 is coupled to the rest support 130 and/or the second support post 114(2) by a table hinge 144 configured to retain the table 142 in an open position substantially perpendicular to the second support post 114(2). The table hinge 144 also allows the table 142 to rotate downward to a closed position in which the table 142 is substantially parallel to the second support post 114(2). Thus, the table 142 is shaped (e.g., with cutouts (as described and illustrated below with reference to FIG. 3)) to prevent engagement with the first and second bottle slider devices 128(1) and 128(2) when the table is moved from the open position to the closed position. Additionally, any shape of the table 142 can be used.

In the open position, the table 142 can be an arm rest for a user in a forward learning position and/or support a breast pump, laptop computer, book, notepad, portable electronic device, or any other items to facilitate multitasking for a user. A user that may desire to rest or sleep without the table 142 in an open position or transport the breast pumping chair system 100 may rotate the table 142 about the table hinge 144 to the closed position. Other positions can also be facilitated by the table hinge 144 and/or other methods for attaching the table 142 to the second support post 14(2) can also be used in other examples.

Accordingly, as illustrated in FIG. 1, to support a user in an ergonomic, comfortable, healthier, forward leaning position, the seat 122 is angled, the first and second leg supports 106(1) and 106(2) are angled partially opposite the seat 122, and the sternum support 132 is angled. The optional abdomen support 134 and headrest support 140 are also angled in substantial alignment with the sternum support 132 to further support the user in the forward learning position.

Advantageously, the breast pumping chair system 100 in this example is highly portable and can be manipulated to a highly compact configuration. For example, the breast pumping assembly 126 can be removed from the chair assembly 102 by decoupling the first and second support posts 114(1) and 114(2), respectively. The first support post 114(1) can be configured to then rotate toward the seat 122 and the second base support 104(2) to facilitate a collapsed position of the breast pumping chair system 100. In some examples, the second base support 104(2) includes a first base support portion 146(1) rotatably coupled to a second base support portion 146(2), the seat 122 is coupled to the first base support portion 146(1), and the second base support portion 146(2) is configured to rotate relative to the first base support portion 146(1) to facilitate the collapsed position.

More specifically, the second base support portion 146(2) can have a hinged connection to the first base support portion 146(1) such that the second foot 112(2) can rotate toward the bottom of the first and second rails 110(1) and 110(2) and under the first and second leg supports 106(1) and 106(2), respectively. In combination, the seat support 124 can be detached from the first base support 104(1) such that the first base support portion 146(1) with the seat 122 can rotate downward toward the first base support 104(1) and between the first and second leg supports 106(1) and 106(2), respectively. With the table 142 and/or the headrest 136 optionally folded and/or detached, the breast pumping chair system 100 can be positioned or folded for maneuverability, optionally facilitated by wheels or castors coupled to one or both of the first foot 112(1) or the second foot 112(2), as explained above.

Optionally, the breast pumping chair system 100 and/or one more portions thereof can be placed in a container or travel case to facilitate transport. Also optionally, the travel case can have a cooler compartment for pumped breastmilk as well as any number of other compartments for a breast pump and/or associated accessories. In yet other examples, the breast pumping chair system 100 can be used for purposes other than breast pumping, such as a medical chair that satisfies particular medical positioning requirements for a patient. In these examples, one or more portions or components of the breast pumping chair system 100 can be made of a radiolucent material to allow a patient to receive chemotherapy, radiotherapy, surgical breast (or other) related medical imaging, and/or in support of any number of other medical procedures and/or patient diagnoses or positioning for spinal taps or other procedures encompassing open posterior spine and patient comfort in any prone or partially prone position.

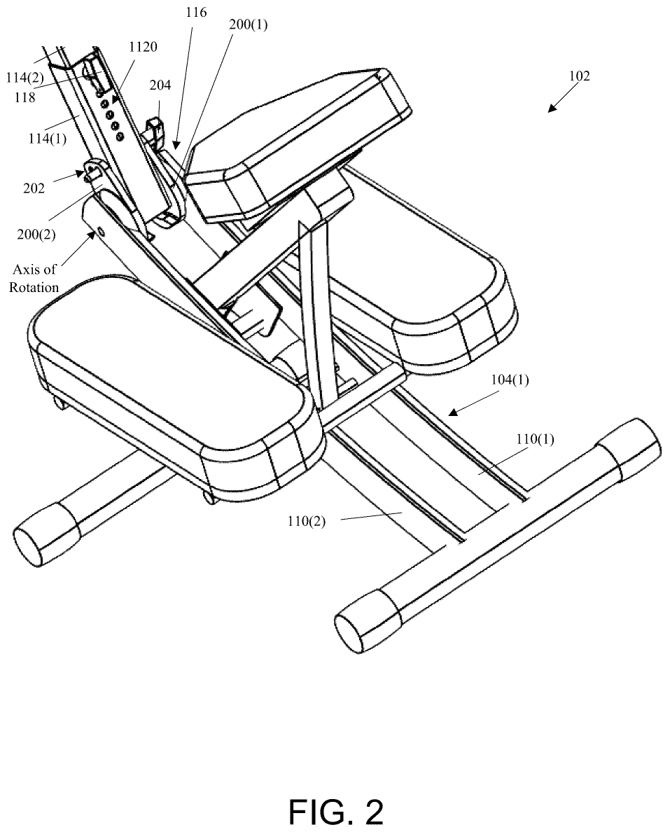

Referring now to FIG. 2, a perspective view of the chair assembly 102 of the breast pumping chair system 100 is illustrated. The chair assembly 102 includes the first support post 114(1) that is coupled to the first base support 104(1) via the post locking mechanism 116. The post locking mechanism 116 includes first and second support plates 200(1) and 100(2) on opposing sides of the first support post 114(1), each of which includes lower apertures (not shown) that facilitate attachment (e.g., via a pin or rod) to the first base support 104(1) to form an axis of rotation of the first support post 114(1) relative to the first base support 104(1).

Each of the first and second support plates 200(1) and 200(2) also includes upper apertures 202 configured to align with side apertures on opposing sides of the first support post 114(1) such that the upper apertures 202 and side apertures are configured to receive a second pin 204 and thereby retain the first support post 114(1) at a desired angle about the axis of rotation. While two upper apertures 202 are illustrated in FIG. 2 to facilitate 45 degrees from prone and 30 degrees forward leaning for a user, any number of upper apertures 202 can be used and/or any number of angles can be facilitated via the post locking mechanism 116 in other examples. With the second pin 204 removed, the first support post 114(1) can rotate between the first and second rails 110(1) and 110(2) of the first base support 104(1) in this example.

As explained above, the first support post 114(1) also has a plurality of through holes 120 configured to retain the second support post 114(2) at a desired height with respect to the first support post 114(1) when the first pin 118 is inserted into one of the through holes 120. Thus, user selection of a particular one of the through holes 120 for insertion of the first pin 118 establishes a comfortable, personalized height of the breast pumping assembly 126 for the user. Other types of retention mechanisms for the angle of the first support post 114(1) and/or the height of the second support post 114(2) can also be used as well as other mechanisms for establishing an angle of rotation for the first support post 114(1).

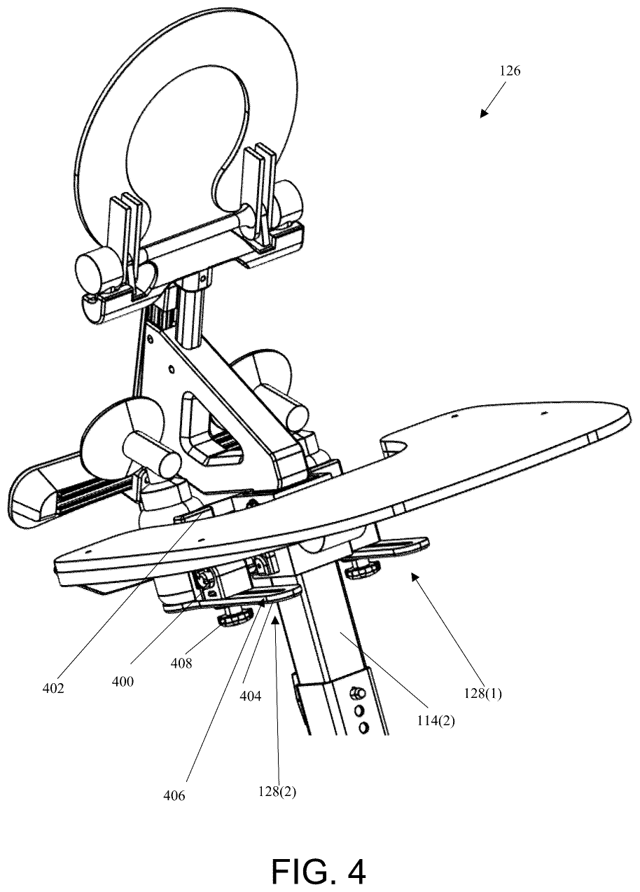

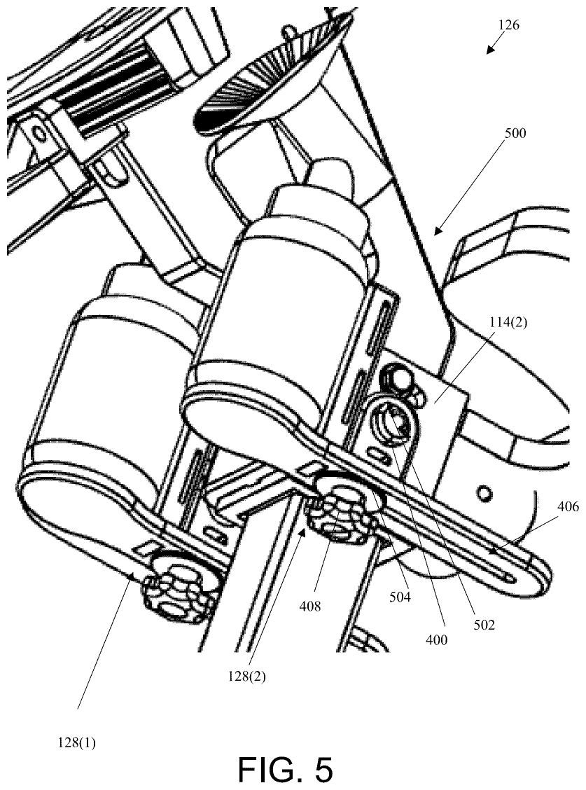

Referring now to FIG. 3, a rear perspective view of the breast pumping assembly 126 of the breast pumping chair system 100 is illustrated. The breast pumping assembly 126 in this example includes the first bottle slider device 128(1) coupled to a side of the second support post 114(2) via a first post extension 300. In some examples, the first bottle slider device 128(1) is the same as the second bottle slider device 128(2) disposed on an opposing side of the second support post 114(2), which is described and illustrated in more detail below with reference to FIG. 4. The first post extension 300 optionally extends upward from a first holder base 302 and facilitates removable attachment to the second support post 114(2) via a rotation guide (described and illustrated with reference to FIG. 5). Thus, the first bottle slider device 128(1) is configured to rotate about the rotation guide to facilitate a desired alignment of a bottle and flange coupled to the first bottle slider device 128(1) for a user. Any number of configurations are possible for successful hands-free breast pumping.

The first bottle slider device 128(1) also includes a first bottle extension 304 that extends upward from the first holder base 302 and is configured to retain a bottle for receiving expressed breastmilk. For example, the bottle can be placed against the first bottle extension 304, supported by the first holder base 302, and straps (not shown) coupled on either end to the first bottle extension 304 can be placed around a portion of the bottle to retain the bottle in place. Other types of retention mechanisms utilizing other types of forces such as compression or magnetic forces, for example, can also be used by the first bottle slider device 128(1) to retain the bottle or any other milk collection system.

The first bottle slider device 128(1) further includes a first track 306 disposed along a portion of a length of the first holder base 302 and through which a first tightening mechanism 308 extends that is configured to retain the first holder base 302 in place at a desired distance from a user and angle of rotation about the first tightening mechanism 308. Any number of tracks may also be used in other examples. The first tightening mechanism 308 can include a tightening nut coupled to a screw or bolt that extends through the first track 306 such that the first holder base 302 can rotate about the screw or bolt and move forward and rearward until reaching a desired location at which point the user can use the first tightening mechanism 308, which may be a pinch and slide or other mechanism, to compress the tightening nut against the first holder base 302 to maintain the first holder base 302 at the desired position. With the first track 306, first tightening mechanism 308, the first bottle extension 304, and the rotation guide, the first bottle slider device 128(1) advantageously facilitates six degrees of movement in three dimensions for individual positioning of the bottle at a desired location for a user.

Also illustrated in FIG. 3 is a cutout 310 in the table 142 that allows the table 142 to move unimpeded by the first bottle slider device 128(1) from an open position to a closed position. The table 142 can include another cutout of the same shape on an opposing side for the same purpose but with respect to the second bottle slider device 128(2). While an exemplary shape of the table 142 is illustrated in FIG. 3, the table 142 can be any shape not limited to oval or rectangular, and/or may have a lip or edge around a perimeter or a portion therefore, and/or can have another configuration in other examples.

Referring now to FIG. 4, a front perspective view of the breast pumping assembly 126 of the breast pumping chair system 100 of FIG. 1 is illustrated. As explained above, the breast pumping assembly 126 can include a second bottle slider device 128(2) that is a mirror image of the first bottle slider device 128(1) with the first and second bottle slider devices 128(1) and 128(2) collectively comprising a bottle adjustment system of the breast pumping assembly 126 of the breast pumping chair system 100. Thus, the second bottle slider device 128(2) is removably coupled to an opposing side of the second support post 114(2) in this example via a second post extension 400 configured to receive a second rotation guide of the second support post 114(2).

The second bottle slider device 128(2) also may include a second bottle extension 402, a second holder base 404, a second track 406, and a second tightening mechanism 408, which can be the same as the first bottle extension 304, first holder base 302, first track 306, and first tightening mechanism 308, although different components can also be used within the first and second bottle slider devices 128(1) and 128(2) in other examples. Thus, the second bottle slider device 128(2) also facilitates six degrees of movement in three dimensions for another bottle coupled thereto for positioning at a desired, comfortable position for a user. Any number of ranges or degrees of movement can be permitted in other examples.

Referring to FIG. 5, a perspective view of an exemplary bottle adjustment system 500 of the breast pumping assembly 126 of FIGS. 3-4 is illustrated. The bottle adjustment system 500 includes the first and second bottle slider devices 128(1) and 128(2) in this example. The second bottle slider device 128(2) is coupled to the second support post 114(2) via the second post extension 400 and the rotation guide 502. Optionally, the rotation guide 502 is configured to extend through an aperture in the second post extension 400 and is shaped in combination with the aperture to allow rotation about only a portion of the circumference of the rotation guide 502 to thereby prevent excessive tipping of an attached bottle and associated spillage of the bottle contents. Any type of retention mechanism can be used to retain the second post extension 400 at a desired degree of rotation about the rotation guide 502 and the second post extension 400 can be engaged with and detached from the second support post 114(2) via any type of locking mechanism.

The second bottle slider device 128(2) also includes the second tightening mechanism 408, which in this example includes a knob attached to a bolt that extends through the second track 406 with a washer 504 on a bottom side of the second track 406 and a nut (not shown) on a top side of the second track 406. Thus, in this example, tightening of the knob within the nut compresses the washer 504 and the nut towards each other to thereby retain the second track 406 at a desired distance from a user and angle about the bolt. However, other types of tightening mechanisms can also be used to restrict movement of the second bottle slider device 128(2) in any number of directions in other examples.

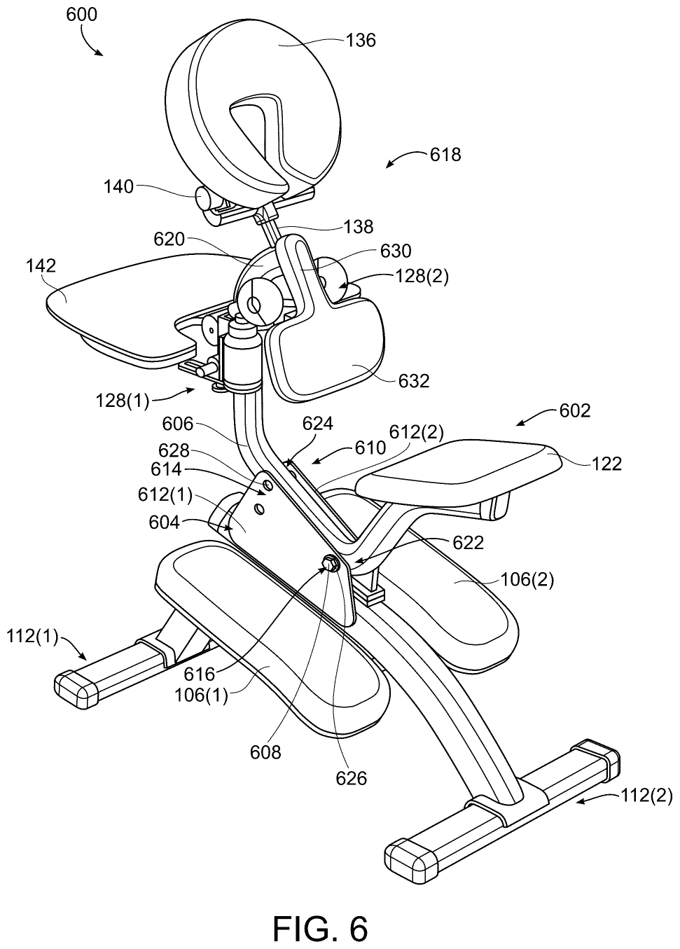

Referring now to FIG. 6, a perspective view of another exemplary breast pumping chair system 600 is illustrated. The breast pumping chair system in this example includes a headrest 136, a headrest support 140, a headrest post 138, a table 142, first and second bottle slider devices 128(1) and 128(2) first and second feet 112(1) and 112(2) a seat 122, and first and second leg supports 106(1) and 106(2) one or more of which can be the same as or different than the corresponding element described and illustrated in more detail above with reference to the breast pumping chair system 100 of FIG. 1. However, the general support structure of the breast pumping chair system 600 illustrated in FIG. 6 is different than that of the breast pumping chair system 100 of FIG. 1.

More specifically, the breast pumping chair system 600 of FIG. 6 includes a chair assembly 602 that has a base frame 604 extending upward from each of the first foot 112(1) and the second foot 112(2) and configured to couple to a seat frame 606 of the chair assembly 602 via a frame hinge 608 and a frame locking mechanism 610. In this example, the base frame 604 includes opposing first and second frame plates 612(1) and 612(2) which can be coupled to the base frame 604 or integral with the base frame 604 when formed as a unitary structure. Each of the first and second frame plates 612(1) and 612(2) includes a plurality of opposing locking apertures 614 and at least one opposing frame plate hinge aperture 616.

The first and second frame plates 612(1) and 612(2) are spaced apart to receive a portion of the seat frame 606. The seat frame 606 is shaped to support the seat 122 at one end and to extend upward toward a breast pumping assembly 618 to receive or be integral with a chest frame 620 at another end. The seat frame 606 in this example includes at least one seat hinge aperture 622 and at least one seat frame locking aperture 624 disposed between the ends of the seat frame 606. The seat hinge aperture 622 and the frame plate hinge aperture 616 are configured to be aligned and to receive a hinge post 626 therethrough to thereby retain the seat frame 606 rotatably coupled to the base frame 604 at the frame hinge 608.

The seat frame locking aperture 624 is configured to selectively align with a pair of the locking apertures 614 on opposing first and second frame plates 612(1) and 612(2) and to receive a locking post 628 therethrough to thereby retain the seat frame 606 in a desired angled position about the frame hinge 608 and with respect to the base frame 604. The locking apertures 614 and locking post 628 collectively form the frame locking mechanism 610 of the chair assembly 602. While two frame plate locking apertures 614 are illustrated in FIG. 6, any number of frame plate locking apertures 614 can be provided in other examples to accommodate any number of angles of the seat frame 606 with respect to the base frame 604. Additionally, washers, clips, ball and socket, and/or any other retaining mechanism(s) can be used to maintain the attachment of the seat frame 606 to the base frame 604 via the frame hinge 608 and frame locking mechanism 610.

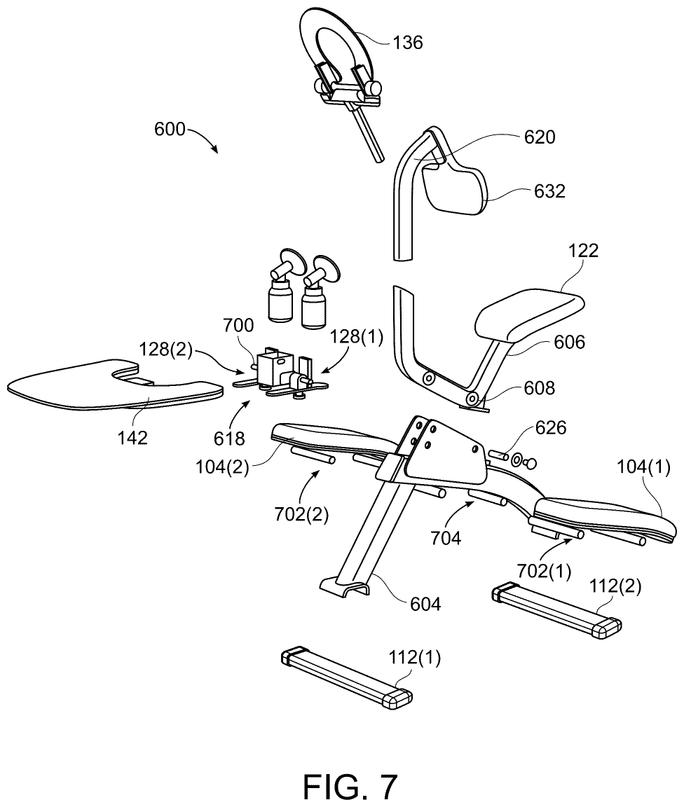

The breast pumping assembly 618 of the breast pumping chair system 600 of FIG. 6 further includes the chest frame 620 that is configured to be attached to or integral with the end of the seat frame 606 that extends upward toward the breast pumping assembly 618. The chest frame 620 can be of a smaller diameter or other dimension to facilitate receipt of one end of the chest frame 620 by the seat frame 606, the chest frame 620 can be of a larger diameter or other dimension to facilitate receipt of one end of the seat frame 606 by the chest frame 620, or any other coupling mechanism can be used in other examples (e.g., a bottle holder support described in detail below with reference to FIG. 7). Optionally, the coupling mechanism for connection of the seat frame 606 and the chest frame 620 allows the breast pumping assembly 618 to move vertically or upward with respect to the chair assembly 602 and lock in place at a height desired by a user. In yet other examples, the seat frame 606 and the chest frame 620 can be formed as a unitary structure. Thus, the chest frame 620 has one end disposed toward the seat frame 606 and another end including a chest support 630 configured to attach to a chest cushion 632 to facilitate support of a user (e.g., in a forward-leaning position).

Referring to FIG. 7, an exploded view of the breast pumping chair system 600 of FIG. 6 is illustrated. In this example, the breast pumping assembly 618 includes a bottle holder support 700 that is sized and configured to receive the chest frame 620 at an upper end and the seat frame 606 at a lower end and to thereby couple those frames together. While a bottle holder support 700 is used to couple the seat frame 606 and the chest frame 620 together in this example, other coupling mechanisms can also be used, and the seat frame 606 and chest frame 620 can also be formed as a unitary structure.

In the example illustrated in FIG. 7, the first and second bottle slider devices 128(1) and 128(2) are coupled to opposing sides of the bottle holder support 700 in a manner the same or similar as discussed above with respect to the rotation guide 502 and first and second post extensions 300 and 400 for example, of the bottle adjustment system 500 of FIG. 5. In other examples, the first and/or second bottle slider device 128(1) or 128(2) can be coupled to the breast pumping chair system 600 in other ways (e.g., directly to the chest frame 620 or the seat frame 606).

Additionally, the first and second leg supports 106(1) and 106(2) in this example optionally include first and second post(s) 702(1) and 702(2) configured to be received by corresponding tubes 704 coupled to a bottom portion of the base frame 604. With the first and second post(s) 702(1) and 702(2) and tubes 704, the first and second leg supports 106(1) and 106(2) can be inserted and removed as desired by a user. Optionally, the first and second post(s) 702(1) and 702(2) can be coupled to the tubes 704 via a locking mechanism and other methods for connecting the first and second leg supports 106(1) and 106(2) to the base frame 604 can also be used, including as explained above with reference to FIG. 1.

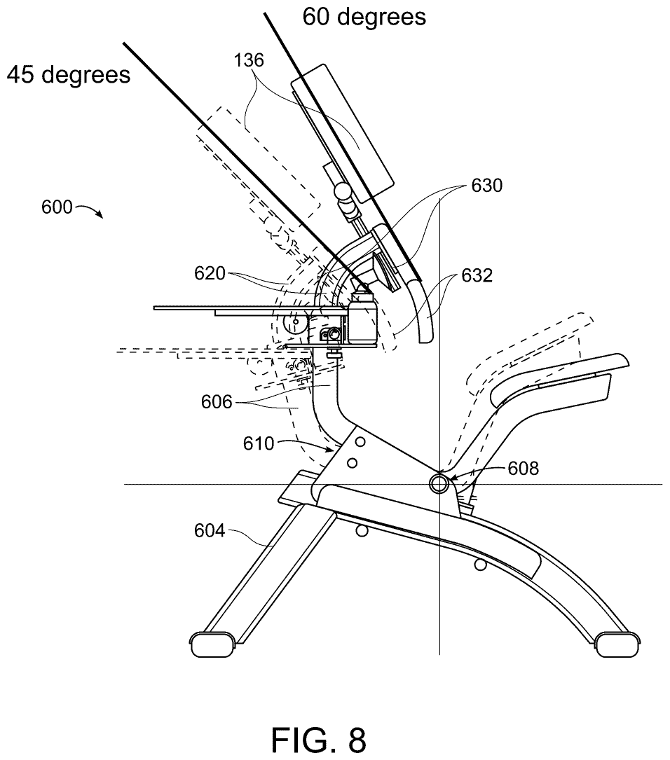

Referring to FIG. 8, a side plan view of the breast pumping chair system 600 of FIG. 6 in two positions facilitated by the base frame 604 is illustrated. Specifically, the two positions illustrated in FIG. 8 correspond to 45 degrees and 30 degrees with respect to the horizontal and vertical axis defined by the frame hinge 608. In particular, at least the chest support 630 disposed proximate an end of the chest frame 620, and optionally one or more of the chest cushion 632 and/or the headrest 136, are substantially aligned at 45 degrees and 30 degrees in the two illustrated positions. The two positions are facilitated by the frame hinge 608 that allows rotation of the seat frame 606 with respect to the base frame 604 and the frame locking mechanism 610, which retains the seat frame 606 in a desired angled position with respect to the base frame 604. Other configurations accommodating any number of other positions and/or angles (e.g., any angle from 0 degrees to 90 degrees) can also be used in other examples.

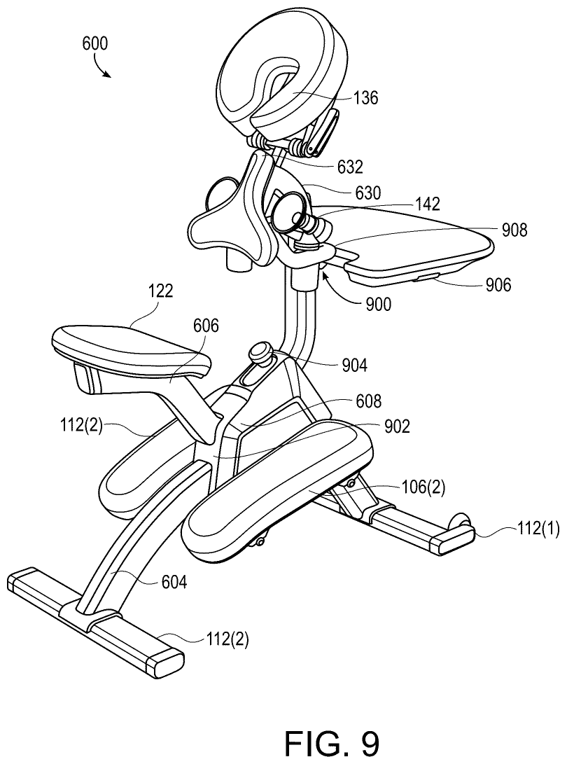

Referring to FIG. 9, a perspective view of the breast pumping chair system 600 of FIG. 6 but with a different exemplary breast pumping assembly 900 is illustrated. In this example, the breast pumping chair system 600 includes a frame cover 902 (e.g., of a plastic material) covering a portion of the seat frame 606 and the base frame 604 proximate the frame hinge 608 and frame locking mechanism 610. A course angle adjustment mechanism 904 protrudes through a top portion of the frame cover 902. The course angle adjustment mechanism 904 can include a knob or other interface device coupled to the frame locking mechanism 610 such that pushing, pulling, or rotating the knob, for example, causes the frame locking mechanism 610 to adjust the seat frame 606 to different angled positions (e.g., the angled positions illustrated in FIG. 8).

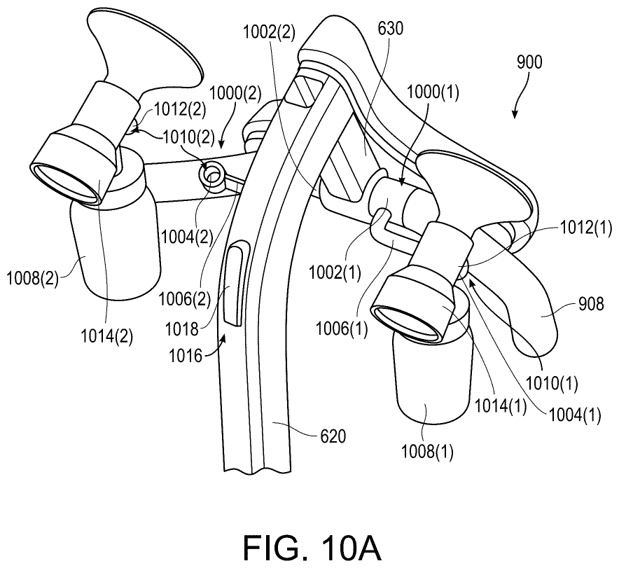

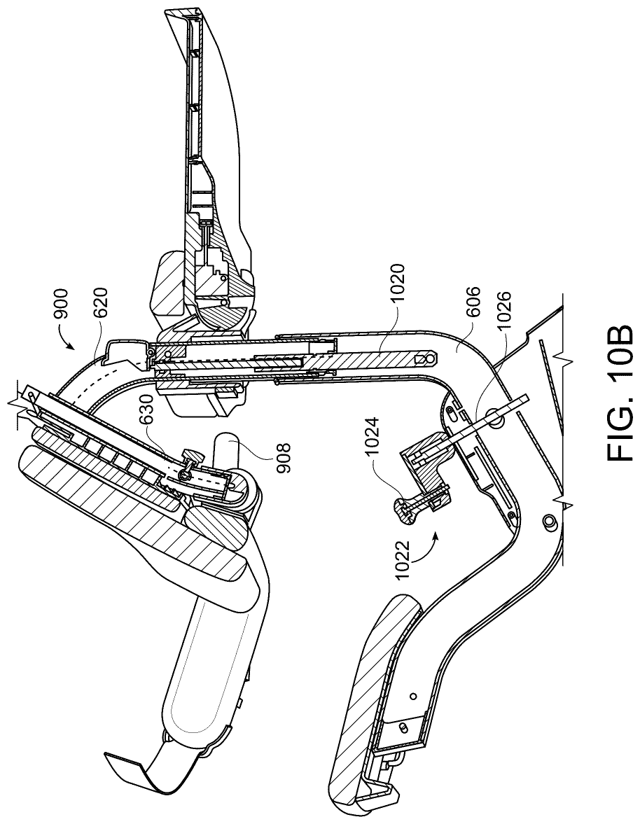

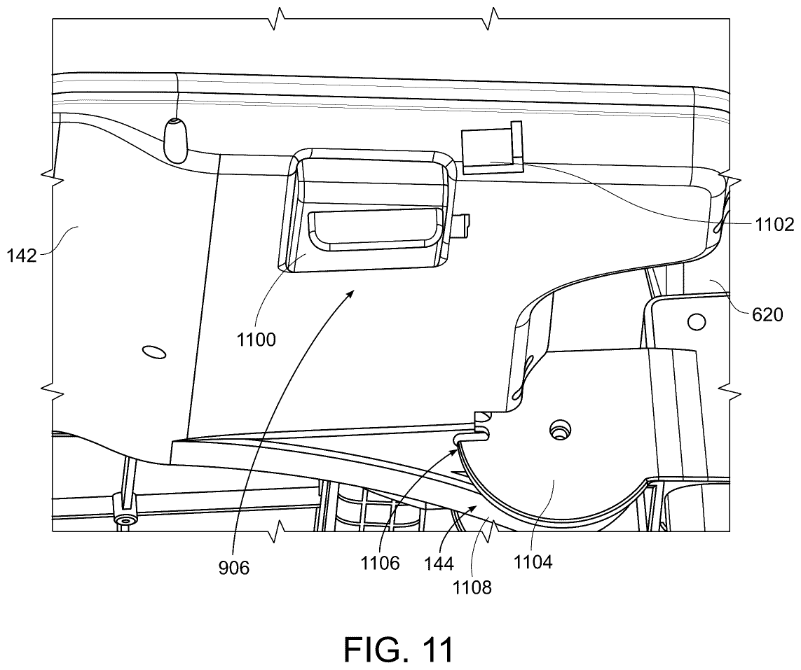

The breast pumping chair system 600 illustrated in FIG. 9 also includes a table adjustment mechanism 906 that facilitates various positions including supported and collapsed or folded positions as explained in more detail below with reference to FIG. 11. In this example, the breast pumping chair system 600 further includes a grab bar 908 that is coupled to the chest support 630 and extends on either side of the chest support 630 to an angled end portion that allows a user to grab or hold as the user is entering, exiting, or using the breast pumping chair system 600. Optionally, the grab bar 908 can move laterally via an aperture in the chest support 630 in other examples. Bottle holder devices are coupled to the grab bar 908 on opposite sides of the chest support 630, as will now be described with reference to FIG. 10.

In FIGS. 10A-B, perspective views of another exemplary breast pumping assembly 900 of the breast pumping chair system 600 of FIG. 9 with angle and height adjustment mechanisms are illustrated. The breast pumping assembly 900 in this example includes the first and second bottle attachment devices 1000(1) and 1000(2) including first and second pivot ends 1002(1) and 1002(2), first and second concave ends 1004(1) and 1004(2), and first and second extensions 1006(1) and 1006(2) disposed between the first and second pivot ends 1002(1) and 1002(2) and the first and second concave ends 1004(1) and 1004(2), respectively. The first and second pivot ends 1002(1) and 1002(2) are disposed around a portion of the grab bar 908 to allow rotational engagement with the grab bar 908.

Additionally, the first and second pivot ends 1002(1) and 1002(2) can move laterally along the grab bar 908 as desired by a user. Thus, the first and second pivot ends 1002(1) and 1002(2) can have a relative snug fit with the grab bar 908 so that the first and second pivot ends 1002(1) and 1002(2), respectively are retained in place during use, and locking and other mechanisms can also be used in other examples. The first and second extensions 1006(1) and 1006(2) can be relatively stiff or at least partially flexible to facilitate more granular adjustment and placement of the first and second bottles 1008(1) and 1008(2), respectively. Further, the first and second extensions 1006(1) and 1006(2) can be coupled to first and second pivot ends 1002(1) and 1002(2) via a mechanism (e.g., first and second ball joint 1010(1) and 1010(2)) that allows for movement in any number of degrees of freedom.

The first and second concave ends 1004(1) and 1004(2) of the first and second bottle attachment devices 1000(1) and 1000(2) are configured to engage with first and second spherical protrusions 1012(1) and 1012(2) of first and second bottle adapters 1014(1) and 1014(2) to thereby form the first and second ball joints 1010(1) and 1010(2) via which the first and second spherical protrusions 1012(1) and 1012(2) are rotatably engaged with first and second concave ends 1004(1) and 1004(2). Other types of ball joints, including with threaded nuts configured to retain the first and second bottle adapters 1014(1) and 1014(2) in a desired position, and other types of connection mechanisms can also be used. The first and second bottle adapters 1014(1) and 1014(2) are configured to be coupled to the first and second bottles 1008(1) and 1008(2) and can be sized to fit any number of breast pumping bottles that include flanges, and/or any other type of breast milk collection system, from any number of manufacturers or suppliers. Thus, any type of breast pump and/or accessories can be used with this technology.

In some examples, irrespective of other size or dimension, the first and second spherical protrusions 1012(1) and 1012(2) are consistent across bottle adapters such that any bottle adapter is configured to engage the first and second concave ends 1004(1) and 1004(2). Thus, the first and second bottle adapters 1014(1) and 1014(2) are configured to rotate within the first and second concave ends 1004(1) and 1004(2) of the first and second bottle attachment devices 1000(1) and 1000(2) to achieve a desired placement of the first and second bottles 1008(1) and 1008(2) and their flanges for a user. While the first and second bottle adapters 1014(1) and 1014(2) are illustrated in FIG. 10 for coupling the first and second bottles 1008(1) and 1008(2) to the first and second bottle attachment devices 1000(1) and 1000(2) clips, ball and socket, straps, and/or any other mechanism can also be used in other examples.

The breast pumping assembly 900 also includes a height adjustment mechanism 1016, which in this example includes an interface 1018 (e.g., a button) coupled to a linear pneumatic actuator 1020. The interface 1018 extends through an aperture of the chest frame 620 within which the linear pneumatic actuator 1020 is disposed. The linear pneumatic actuator 1020 can be coupled at a lower portion to the base frame 604 and, at an upper portion, to the chest frame 620. Thus, a user can engage the interface 1018 to cause the linear pneumatic actuator 1020 to move linearly thereby moving the chest frame 620 coupled thereto in an upward direction with respect to the base frame 604 (e.g., within the base frame 604). Once the user has achieved the desired height of the breast pumping assembly 900, disengagement of the interface 1018 causes the linear pneumatic actuator 1020 to retain its position. Other methods of facilitating linear (i.e., upward and downward) motion of the breast pumping assembly 900 can also be used in other examples.

Referring more specifically to FIG. 10B, a granular angle adjustment mechanism 1022 is illustrated that includes a crank handle 1024 coupled to an angle adjustment post 1024. The angle adjustment post 1026 can be threaded within one or both of the crank handle 1024 and/or seat frame 606 such that rotation of the crank handle 1022 alternately lowers or raises the angle adjustment post 1024 to thereby alter the angle of the breast pumping assembly 900 about the frame hinge 608, and retention of the seat frame 606 in place, as desired by a user. Other types of angle adjustment mechanisms can also be used in other examples.

Referring to FIG. 11, the table adjustment mechanism 906 of the breast pumping chair system 600 of FIG. 9 is illustrated. In this example, the table adjustment mechanism 906 includes the table hinge 144, a puller 1100, and a lock 1102. The table hinge 144 includes an external table bracket 1104 with a plurality of detents 1106, optionally or varying size (e.g., depth). The external table bracket 1104 is removably coupled to the chest frame 620 in this example. The table hinge 144 also includes an internal table bracket 1108 coupled to the table 142 at one end and rotatably coupled to the external table bracket 1104 at another end (e.g., via a rod or axle disposed proximate a central portion of one or more of the internal table bracket 1108 and the external table bracket 1104. Thus, the external table bracket 1104 can extend on both sides of the internal table bracket 1108 in some examples.

In use, the puller 1100 can be engaged by a user to release a support (not shown) coupled thereto, disposed within the table 142, and configured to engage one of the detents 1106 when released. Optionally, at least a lowermost one of the detents 1106 extends further away from the chest frame 620 to prevent an undesired drop of the table 142. The lock 1102 is coupled via another support (not shown) to the table hinge 144 such that rotation of the internal table bracket 1108 about the external table bracket 1104 is restricted beyond a predetermined angle when the lock 1102 is not engaged. Thus, a user with can pull the puller 1100 and push the lock 1102 rearward to allow the table 142 to fully rotate to a closed position. Other typers of adjustment mechanisms can also be used in other examples.

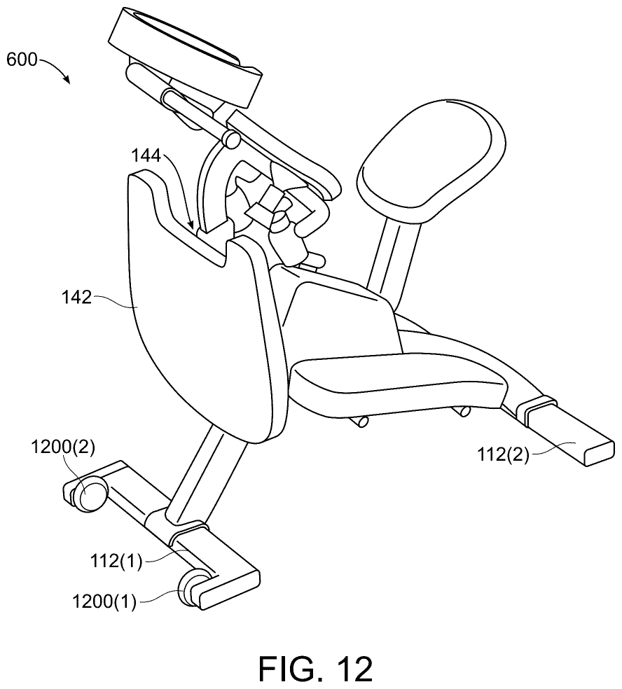

Referring now to FIG. 12, a perspective view of the breast pumping chair system 600 of FIG. 9 with the table 142 in a collapsed position is illustrated. The table 142 can be rotated about the table hinge 144 (e.g., a locking hinge) to a collapsed position as illustrated in FIG. 12 using the table adjustment mechanism 906, for example. In this example, the first foot 112(1) includes first and second wheels 1200(1) and 1200(2) that, along with the collapsible table 142, facilitate efficient maneuverability and/or storage of the breast pumping chair system 600. The first and second wheels 1200(1) and 1200(2) can be locking wheels and can be included on one or both of the first or second feet 112(1) or 112(2) in some examples.

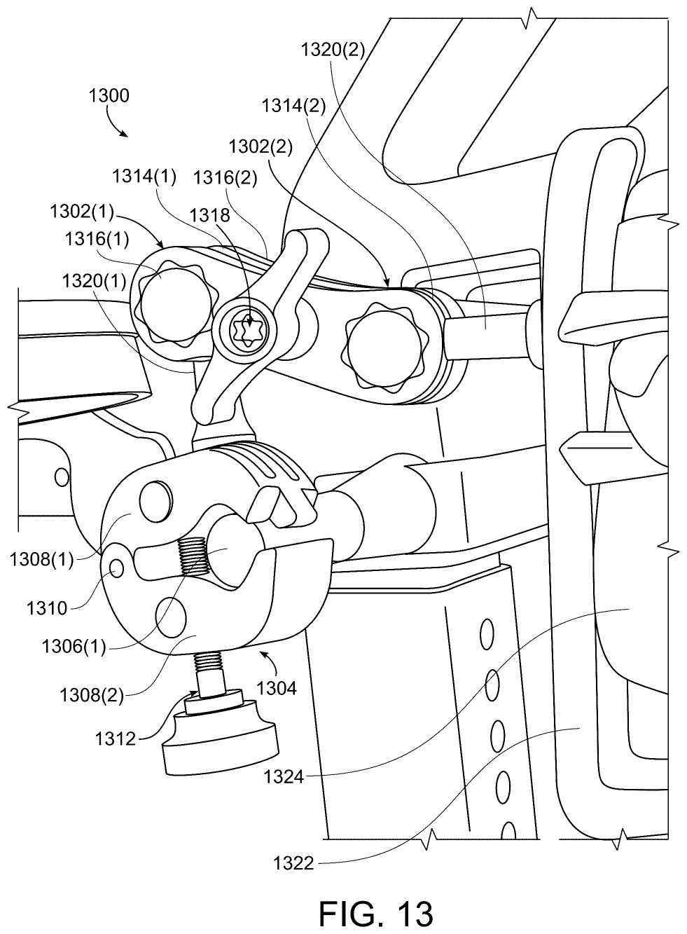

Referring now to FIG. 13, a perspective view of an exemplary milk collection system 1300 with first and second ball joints 1302(1) and 1302(2) coupled to a clamp 1304 attached to a first collection system post 1306(1) is illustrated. While only one milk collection system 1300 is illustrated and described herein, two opposing milk collection systems can be provided in other examples. The clamp 1304 in this example is a vice-like component with upper and lower portions 1308(1) and 1308(2) coupled together at hinge 1310, although other types of clamps can also be used in other examples. The clamp 1304 is configured to attach to the first collection system post 1306(1), which can be the same as or different than the first and second post extensions 300 and 400 of FIGS. 3 and 4, for example. The clamp 1304 can be configured with a first tightening mechanism 1312 configured to reduce the inner diameter of the clamp 1304 when tightened for attachment to the first collection system post 1306(1).

The milk collection system 1300 includes first and second ball joints 1302(1) and 1302(2) with first and second balls 1314(1) and 1314(2) contained within a housing that has first and second opposing sides 1316(1) and 1316(2) and a second tightening mechanism 1318 coupled to the first and second opposing sides 1316(1) and 1316(2). The first ball joint 1302(1) is attached to the clamp 1304 via a first holder post 1320(1) coupled to the first ball 1314(1) and the second ball joint 1302(2) is attached to a bottle holder 1322 via a second holder post 1320(2) coupled to the second ball 1314(2).

In some examples, the second tightening mechanism 1318 is coupled to the first and second opposing sides 1316(1) and 1316(2) such that in a loose state, the first and second balls 1314(1) and 1314(2) may or may not be removed from within the ball joint housing. In a tightened state, the second tightening mechanism 1318 applies pressure from the first and second opposing sides 1316(1) and 1316(2) to the first and second balls 1314(1) and 1314(2) to thereby retain the first and second balls 1314(1) and 1314(2), as well as the bottle holder 1322 coupled to the second ball 1314(2) via the second holder post 1320(2), in a desired position. Each of the first and second ball joints 1302(1) and 1302(2) in this example advantageously has at least three degrees of rotational freedom for placement of the bottle holder 1322, and bottle 1324 attached thereto, in a desired location by a user.

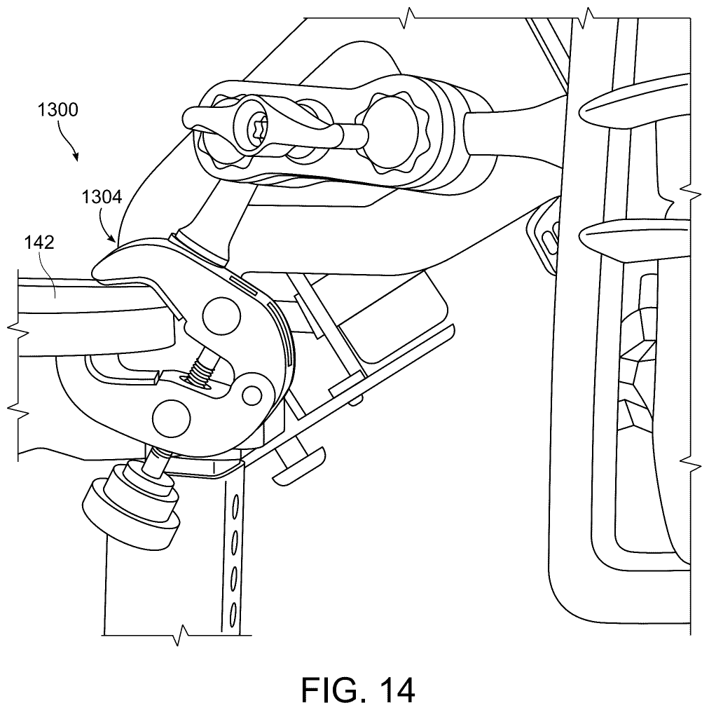

Referring to FIG. 14, a perspective view of the exemplary milk collection system 1300 of FIG. 13 with the clamp 1304 coupled to the table 142 is illustrated. While the clamp 1304 can be shaped with an inner diameter to receive the first collection system post 1306(1), as illustrated in FIG. 13, in other examples, the clamp 1304 can be configured to attached to a counter, table, kitchen island, desk, or any other surface having an overhang, for example. Thus, the milk collection system 1300 described and illustrated by way of the examples herein could be used with a system that includes a chest support 630 with a chest cushion 632 that accepts a headrest 136 or with another type of breast pumping system that attaches to a surface, for example.

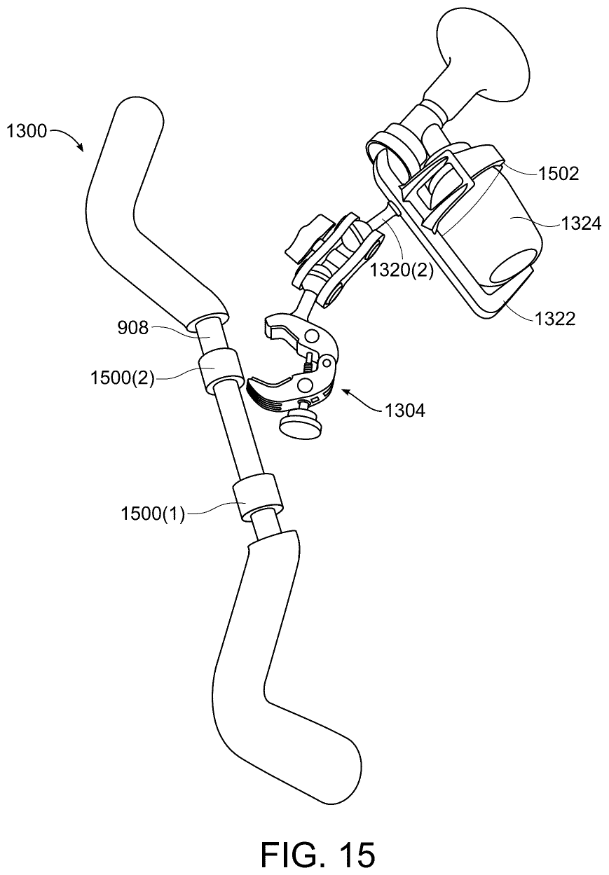

Referring to FIG. 15, a side plan view of the milk collection system 1300 of FIG. 13 with the clamp 1304 disposed proximate first and second slider attachments 1500(1) and 1500(2) of the milk collection system 1300, which is coupled to the grab bar 908, is illustrated. The grab bar 908 can be the same as or different than the grab bar illustrated in FIG. 9, although another type of handle or other bar can also be used in other examples. In this example, the clamp 1304 is configured to attach to one of the first and second slider attachments 1500(1) and 1500(2), which can be via another ball joint, a snap, a clip, a strap, or any other type of connection The first and second slider attachments 1500(1) and 1500(2) are slidably coupled to the grab bar 908 to allow at least lateral movement. Optionally, the first and second slider attachments 1500(1) and 1500(2) also facilitate rotational movement around at least a portion of the grab bar 908.

The bottle 1324 is illustrated in this example received by the bottle holder 1322. The bottle holder 1322 in this example includes an adjustable strap 1502 configured to securely hold bottles having varying size (e.g., between 4 ounces and 20 ounces) and varying thickness. Other types of bottle holders can also be coupled to the second holder post 1320(2) in other examples, including the bottle holders described in more detail above and below.

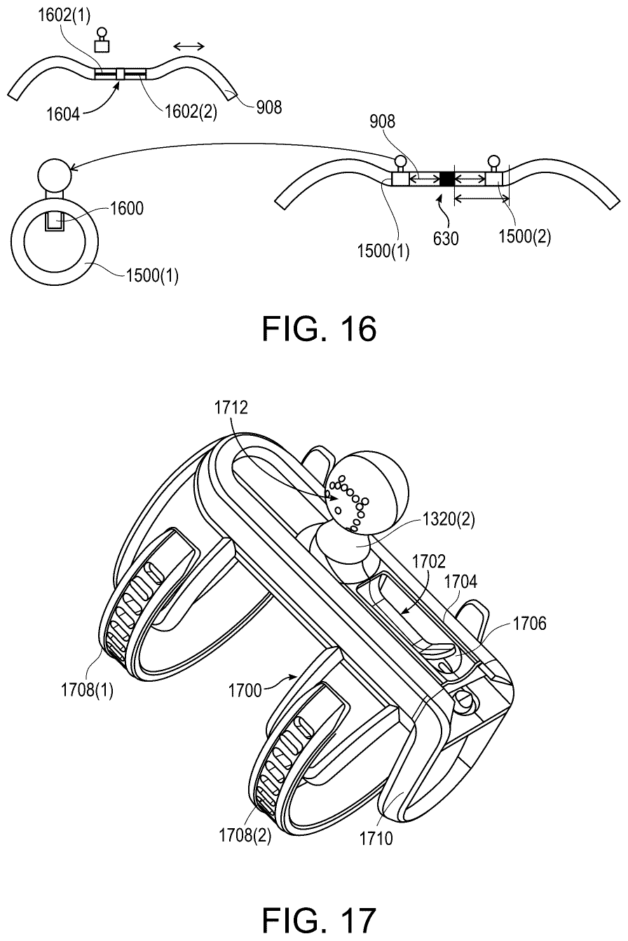

Referring to FIG. 16, a side plan view of the milk collection system 1300 of FIG. 15 illustrating the attachment of the first and second slider attachments 1500(1) and 1500(2) is illustrated. In this example, the first slider attachment 1500(1) includes a notch 1600 configured to be received by a first slot 1602(1), slide, or track of the grab bar 908. In this example, the grab bar 908 includes first and second slots 1602(1) and 1602(2) disposed on either side of a chest support 630 to which the chest cushion 632 is coupled. The notch 1600 and the first slot 1602(1) collectively restrict the rotational movement of the first slider attachment 1500(1) around the grab bar 908 as well as the lateral movement of the first slider attachment 1500(1) along a long axis of the first slot 1602(1). In other examples, the first slider attachment 1500(1) can have a clamp or other type of mechanism for selective coupling to the grab bar 908 in a fixed position.

Referring to FIG. 17, a perspective view of another exemplary bottle holder 1700 is illustrated. The bottle holder 1700 in this example includes the second holder post 1320(2) coupled to a vertical slider mechanism 1702 configured to slide vertically in a track 1704 disposed on a rear of the bottle holder 1700. The vertical slider mechanism 1702 includes a protrusion 1706 disposed at an end opposite the second holder post 1320(2) that can be engaged by a user to facilitate the movement of the vertical slider mechanism 1702 and the second holder post 1320(2) coupled thereto within the track 1704. Thus, when the vertical slider mechanism 1702 is moved within the track 1704, the bottle holder 1700 and bottle retained therein can be moved to a desired vertical position by a user.

In this example, the bottle holder includes upper and lower straps 1708(1) and 1708(2) with clasps to retain the bottle in place supported by a bottle floor 1710, although other methods for retaining the bottle in place can also be used. Additionally, the second ball 1314(2) includes a plurality of indentations 1712 that, when engaged with mirrored protrusion(s) (not shown) (e.g., in one or both of first and second opposing sides 1316(1) and 1316(2) of the second ball joint 1302(2)) allow a user to retain the bottle holder 1700 at a desired angle of rotation. The location of the protrusions and indentations 1712 can be reversed in other examples. Additionally, the productions and indentations 1712 can be provided on one or both of the first or second ball joints 1302(1) and 1302(2). Other methods can also be provided to maintain the bottle holder 1700 at a desired vertical height and/or angle, including those described and illustrated herein with reference to other examples.

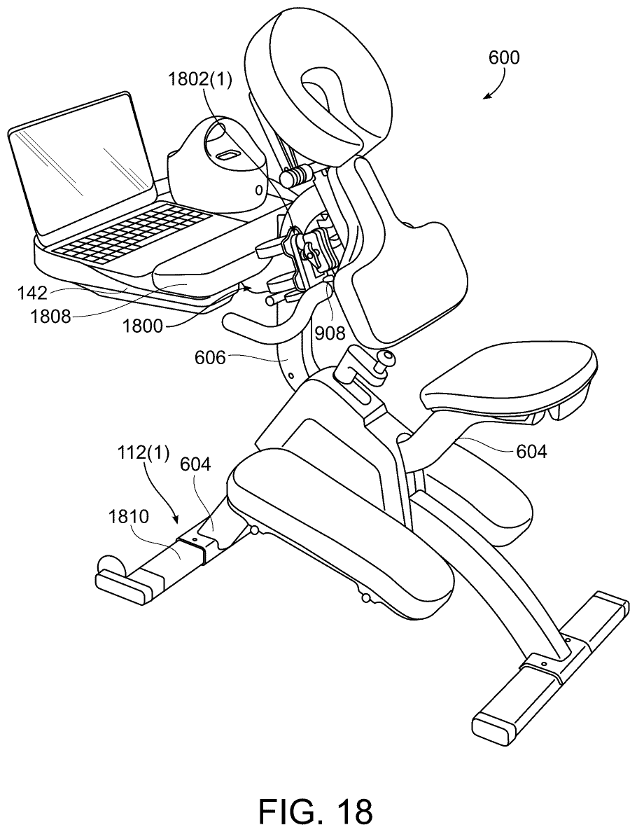

Referring now to FIG. 18, a perspective view of the breast pumping chair system 600 of FIGS. 9 and 11 with another exemplary milk collection system 1800 including another exemplary first bottle holder 1802(1) is illustrated. While only the first bottle holder 1802(1) is illustrated in FIG. 18, a second bottle holder 1802(2) (not shown in FIG. 18) could be coupled to the grab bar 908 in other examples explained in more detail below. In those examples, the second bottle holder 1802(2) could be the same as, or a mirror image of, the first bottle holder 1802(1). Thus, the milk collection system 1800 in other examples can include both the first and second bottle holders 1802(1) and 1802(2).

The breast pumping chair system 600 in the example illustrated in FIG. 18 includes additional optional components including an arm rest 1808 disposed on a top of the table 142, which in this example includes a lip or rim to prevent materials (e.g., a breast pump) from sliding off. Optionally, one or more elastic bands can be used to retain the arm rest 1808 substantially in place when the table 142 is lowered to a closed position. Hook and loop fasteners and/or other types of retention systems can also be used to retain the arm rest 1808 in place in other examples. A pad 1810 (e.g., made of silicone) is also provided as a footrest on the first foot 112(1). While only one pad 1810 is illustrated in FIG. 18, multiple pads can be provided on the first foot 112(1) (e.g., on both sides of the intersection of the base frame 604 with the first foot 112(1)).

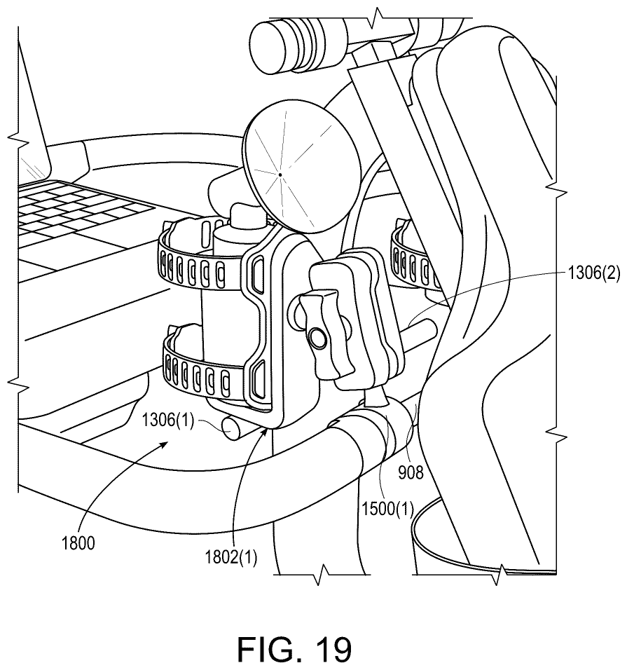

Referring to FIG. 19, a perspective view of the milk collection system 1800 of FIG. 18 attached to the grab bar 908 is illustrated. In this example, the first slider attachment 1500(1) is coupled to the grab bar 908 and the first bottle holder 1802(1). Although the milk collection system 1800 is illustrated in FIGS. 18 and 19 as coupled to the grab bar 908, the milk collection system 1800, as well as any other of the milk collection systems described and illustrated herein, can also be coupled to the first and/or second collection system posts 1306(1) and 1306(2) in other examples.

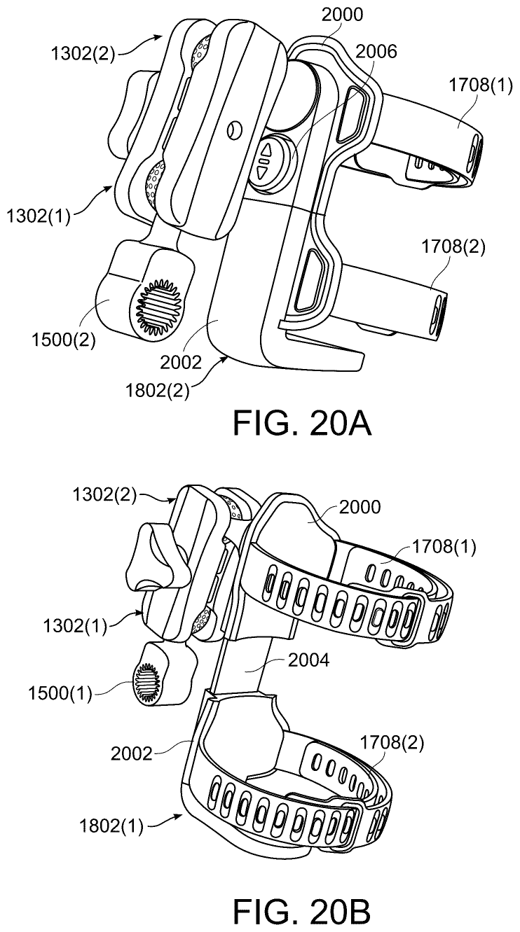

Referring to FIGS. 20A-B, rear and front perspective views of the second bottle holder 1802(2) and the first bottle holder 1802(1), respectively, of the milk collection system 1800 of FIG. 18 are illustrated. In this example, the milk collection system 1800 is configured to operate similar to or the same as the milk collection system 1300 with respect to the first and second ball joints 1302(1) and 1302(2) but includes the first and second slider attachments 1500(1) and 1500(2). Other permutations of the various components of any of the milk collection systems described and illustrated herein can also be used in other examples.

The first and second bottle holders 1802(1) and 1802(2) in this example also include upper and lower components 2000 and 2002 that are coupled together via a holder bar 2004 that is configured to be received by the upper and lower components 2000 and 2002. The holder bar 2004 is coupled to a holder button 2006 such that engagement with the holder button 2006 by a user allows free movement of the upper and lower components 2000 and 2002 about the holder bar 2004 and with respect to each other. Thus, disengagement of the holder button 2006 restricts movement of the upper and lower components 2000 and 2002 about the holder bar 2004 and with respect to each other. In this example, the upper strap 1708(1) is coupled to the upper component 2000 and the lower strap 1708(2) is coupled to the lower component 2002. Thus, FIG. 20B illustrates an extended position of the first bottle holder 1802(1) as compared to that of FIG. 20A, which accommodates and more effectively secures larger bottles and/or facilitates a desired vertical placement of a bottle held by the first bottle holder 1802(1).

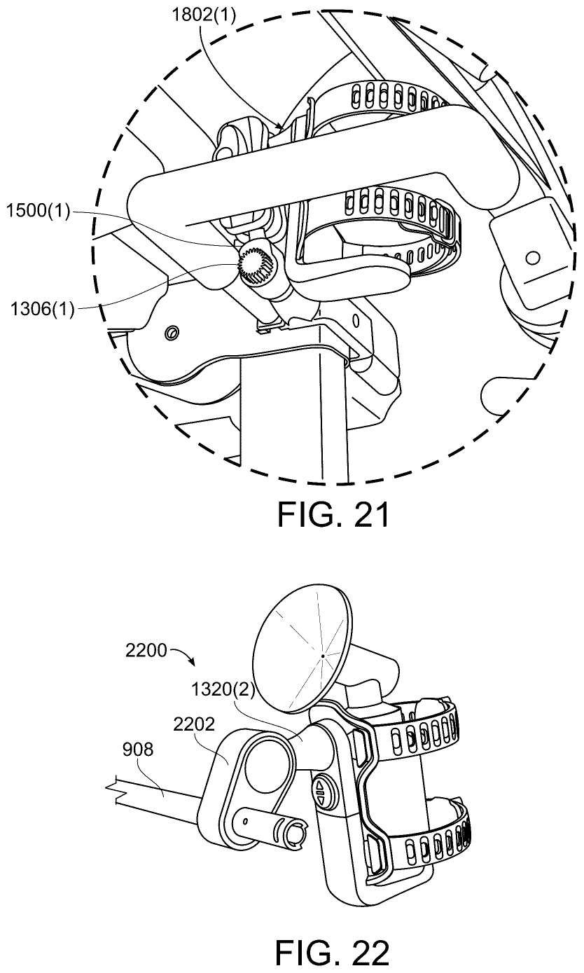

Referring to FIG. 21, a perspective view of the milk collection system 1800 of FIG. 18 coupled to the first collection system post 1306(1) is illustrated. As explained above, any of the milk collection systems disclosed herein can be coupled to the first and/or second collection system posts 1306(1) and 1306(2) and/or the grab bar 908. In the example illustrated in FIG. 21, the first slider attachment 1500(1) is configured to receive and be retained by the first collection system post 1306(1). Optionally, the first collection system post 1306(1) includes the first slot 1602(1), for example, to prevent rotation and/or lateral movement that would otherwise disengage the first slider attachment 1500(1) from the first collection system post 1306(1).

Referring to FIG. 22, a perspective view of a portion of another exemplary milk collection system 2200 with a ball joint housing 2202 is illustrated. The milk collection system 2200 on this example can be the same as the milk collection system 1800 of FIG. 18 except that it includes the ball joint housing 2202 and excludes any slider attachments (e.g., first and second slider attachments 1500(1) and 1500(2)). The ball joint housing 2202 can include an aperture for receiving the grab bar 908 (or the first and/or second collection system posts 1306(1) and 1306(2) in other examples) and can surround the second holder post 1320(2). Optionally, the ball joint housing has a notch (e.g., notch 1600) or shape on an internal portion to prevent rotation and/or lateral movement that would otherwise disengage the first slider attachment 1500(1) from the grab bar 908. Accordingly, the ball joint housing 2202 can move laterally along, and/or rotate about, the grab bar 908 in this example to achieve a location desired by a user.

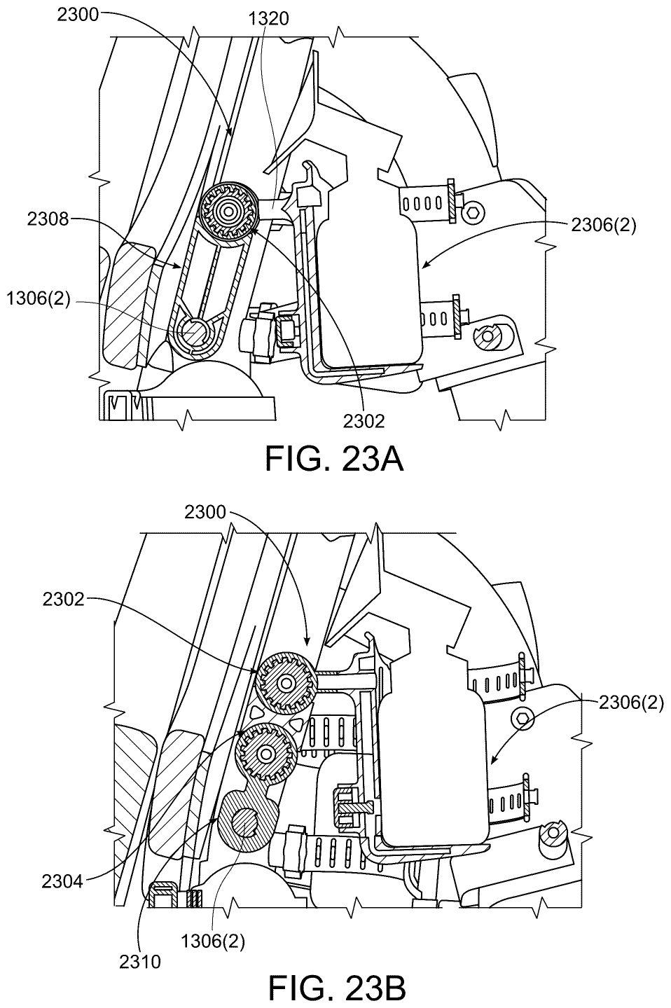

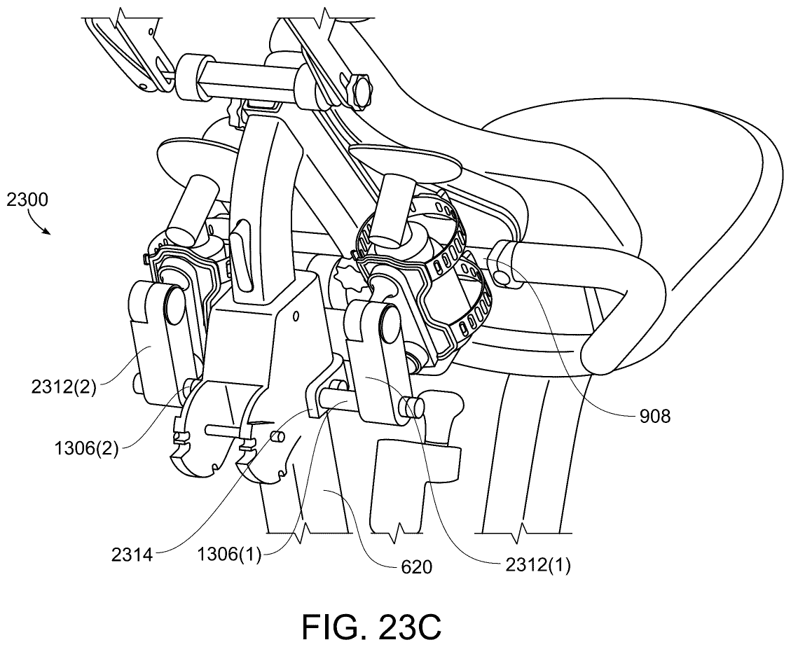

Referring to FIGS. 23A-C, perspective views of another exemplary milk collection system 2300 with first and/or second gears 2302 and 2304 are illustrated. The milk collection system 2300 in this example includes first and second bottle holders 2306(1) and 2306(2), which can be the same as the first and second bottle holders 1802(1) and 1802(2) except that the first and second holder posts 1320(1) and 1320(2) are coupled to the first gear 2302. Thus, while the first and second bottle holders 1802(1) and 1802(2) can be rotatably coupled to the grab bar 908 or the first and second collection system posts 1306(1) and 1306(2) via first and/or second ball joints 1302(1) and 1302(2), as explained in detail above, in the examples illustrated in FIGS. 23A-C, the first and second bottle holders 2306(1) and 2306(2) are rotatably coupled to the first and second collection system posts 1306(1) and 1306(2) via first and/or second gears 2302 and 2304.

Referring more specifically to FIG. 23A, the second bottle holder 2306(2) includes the second holder post 1320(2) coupled to the first gear 2302, which is coupled to a single gear frame 2308. The single gear frame 2308 is configured to receive the second collection system post 1306(2) toward a first end and engage with the first gear 2302 toward a second end such that the first gear 2302 can rotate and lock the second bottle holder 2306(2) at an angle and position desired by a user (as well as unlock to facilitate alternate rotation positions). Optionally, the second collection system post 1306(2) and the single gear frame 2308 can be shaped so as to restrict rotation of the single gear frame 2308 about the second collection system post 1306(2) when the single gear frame 2308 is attached to the second collection system post 1306(2).

Referring more specifically to FIG. 23B, the second bottle holder 2306(2) in this example also includes the second holder post 1320(2) coupled to the first gear 2302, but the first gear 2302 is coupled to a double gear frame 2310. The first gear 2302 engages the double gear frame 2310 in this example in the same manner as described above with respect to the single gear frame 2308. However, the double gear frame 2310 is further configured to rotatably engage the second gear 2304 to facilitate additional rotational positions and degrees of freedom for users. The double gear frame 2310 can also be configured to attach to the second collection system post 1306(2) in the same manner as described above with reference to the single gear frame 2308. Additionally, any other number of gears can be used in other examples and other methods for rotatably attaching the first and/or second bottle holders 2306(1) and 2306(1) of the milk collection system 2300 to the grab bar 908 and/or first and/or second collection system posts 1306(1) and 1306(2) can also be used in other examples.

Referring more specifically to FIG. 23C, the milk collection system 2300 is illustrated with first and second gear housings 2312(1) and 2312(2) within which first and second single gear frames (not shown) and first and second first gears (not shown) are contained. The milk collection system 2300 also includes a collection system bracket 2314 configured to be removably attached to the chest frame 620 and coupled to, and/or integrally including, the first and second collection system posts 1306(1) and 1306(2). Accordingly, a user can optionally remove the collection system bracket 2314 with attached first and second collection systems posts 1306(1) and 1306(2) as desired (e.g., when using the milk collection system 2300 as attached to the grab bar 908).

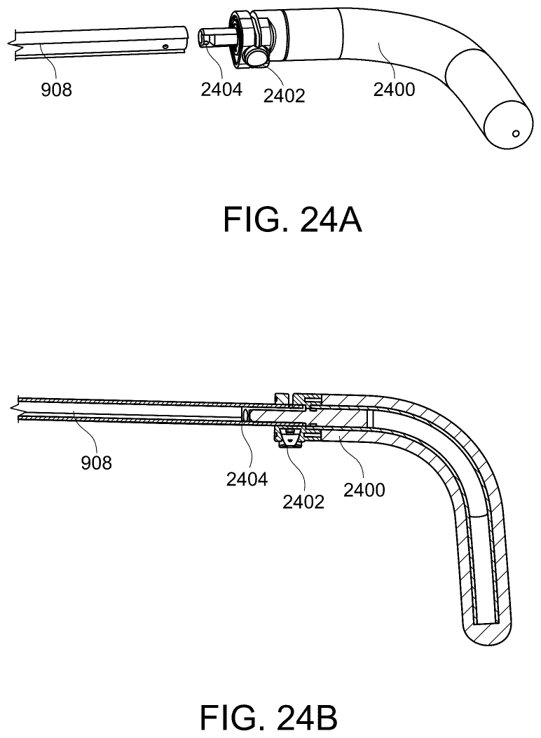

Referring to FIGS. 24A-B, perspective and plan views of an exemplary handle 2400 detached from, and attached to, respectively, the grab bar 908 are illustrated. In some examples, the handle 2400 can be removed from the grab bar 908 (e.g., to facilitate attachment of a portion of milk collection system 2300) and/or the grab bar 908 itself can be removed from the breast pumping chair system 600 (e.g., when the milk collection system 2300 is used with the first and second collection system posts 1306(1) and 1306(2)). While only one handle 2400 is illustrated in this example, another handle (not shown) can be disposed on an opposite end of the grab bar 908 and can be a mirror image of the handle 2400.

In this example, the handle 2400 includes a handle button 2402 that is configured to engage a locking component 2404 to thereby disengage the locking component from the grab bar 908, and thereby facilitate removal of the handle 2400 from the grab bar 908. More specifically, the locking component 2404 is configured to engage with the grab bar 908 when the grab bar 908 is inserted into the handle 2400. A user can then press the handle button 2402 to engage the locking component 2404 causing the locking component 2404 to disengage from the grab bar 908 and allowing the user to pull the handle 2400 out of and away from the grab bar 908. Other methods for removably attaching the handle 2400 to the grab bar 908 can also be used in other examples.

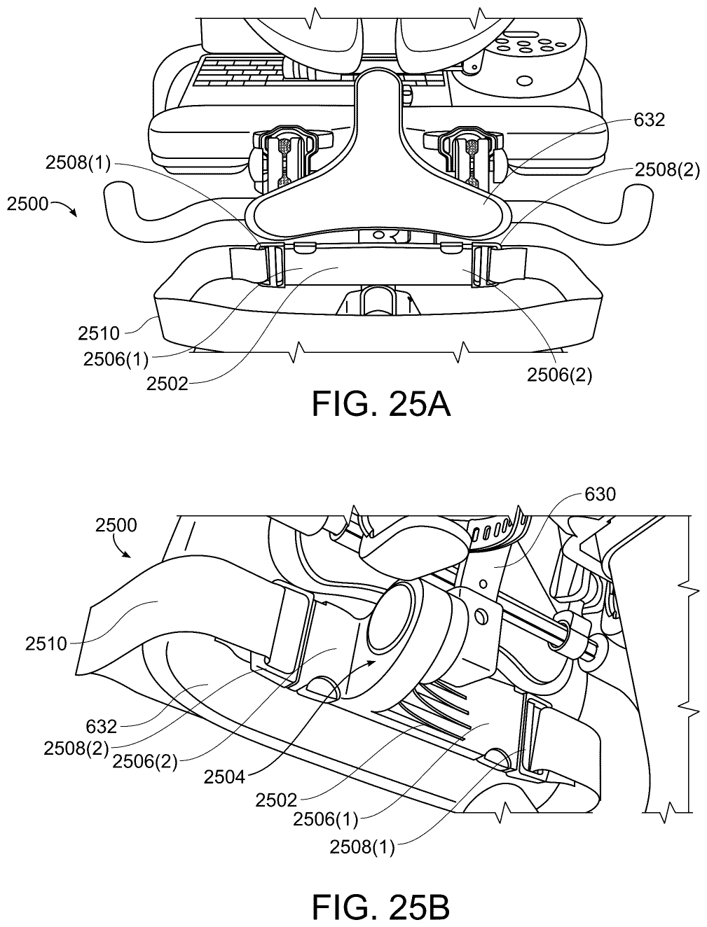

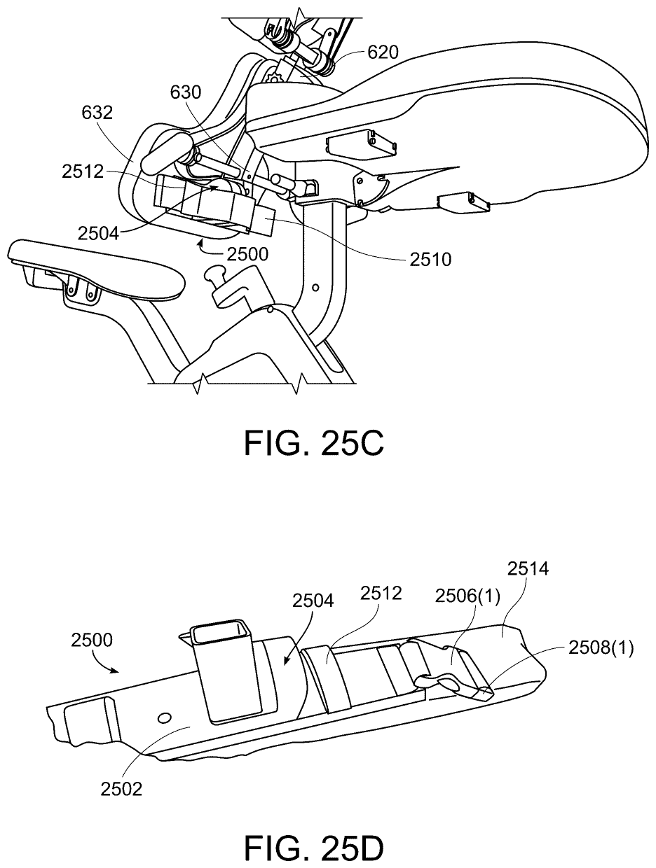

Referring now to FIGS. 25A-D, an exemplary seatbelt system 2500 is illustrated. The seatbelt system 2500 in this example includes a pelvic support 2502 attached to the chest support 630 below or proximate a bottom portion of the chest cushion 632, although the pelvic support can be coupled to other components (e.g., the chest frame 620) and/or disposed in other locations. Optionally, the pelvic support 2502 is attached to the chest support 630 via a rotatable attachment mechanism 2504. The pelvic support 2502 includes first and second female attachment mechanisms 2506(1) and 2506(2) (e.g., buckle, clip, or clasp) disposed at opposing ends and configured to engage male attachment mechanisms 2508(1) and 2508(2) that are coupled to opposing ends of a webbing 2510.

Accordingly, the webbing 2510 can be extended around a user's body, tightened, and clasped at both ends to provide a more secure, relaxed engagement with the breast pumping chair system 600. In some examples, a rear portion of the pelvic support 2502 includes one or more straps 2512 to retain a portion of the webbing when the seatbelt system 2500 is not in use. Also optionally, the pelvic support 2502 can include a pelvic cushion 2514 for more comfortable usage of the seatbelt system 2500. Other types of straps, belts, and/or connections can also be used in other examples.

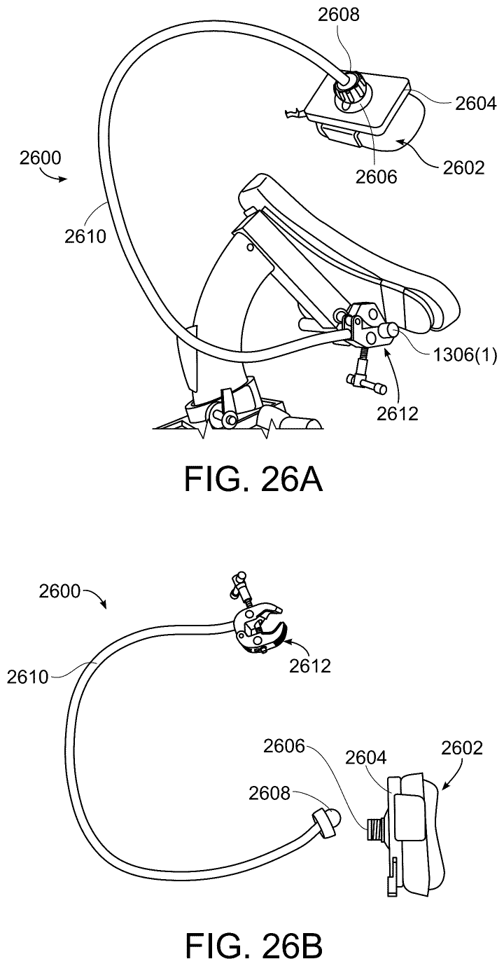

Referring to FIGS. 26A-B, an exemplary accessory system 2600 is illustrated. In this example, the accessory system 2600 includes a warming and/or vibrating device 2602, which can generate heat and/or vibration to facilitate improved milk expression when in use. The accessory system 2600 is retained by an accessory bracket 2604 with a socket 2606 on a rear portion. The socket 2606 is configured to engage a ball 2608 to form a ball 2608 and socket 2606 joint that allows rotation of the warming and/or vibrating device 2602 to a desired position by a user. The ball 2608 is disposed at one end of a flexible, rigid, and/or adjustable arm 2610 that has a stiffness that allows the arm 2610 to retain its position after manipulation by a user. At another end of the arm 2610 opposite the ball, the arm 2610 is coupled to an arm connection mechanism 2612, which can be the same or different as the clamp 1304, for example. The arm connection mechanism 2612 facilitates attachment to the first connection system post 1306(1) in this example, although the arm 2610 can be connected in other manners and/or to other components of the breast pumping chair system 600. Additionally, different types of connections other than a ball and socket joint between the arm 2610 and the accessory bracket 2604 can also be used in other examples.

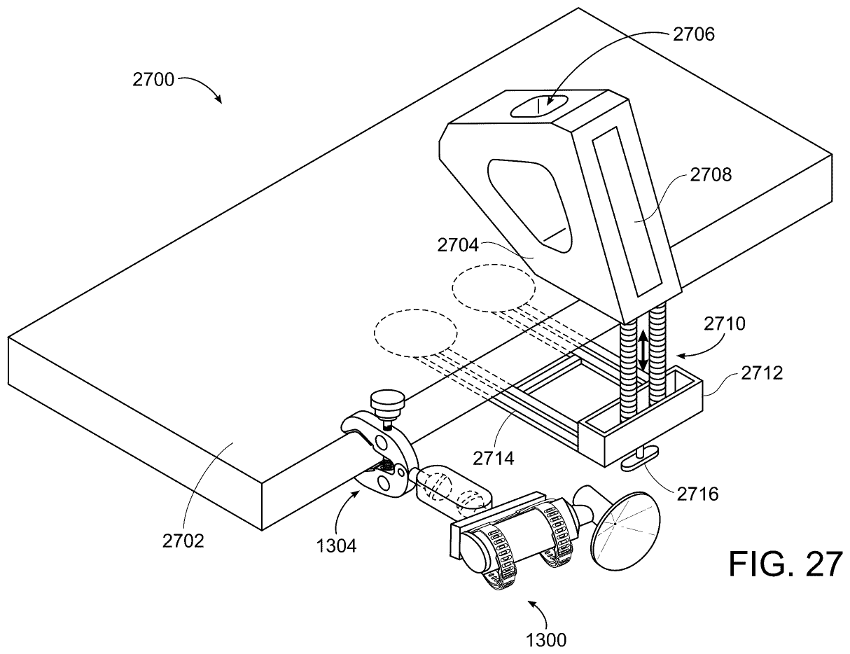

Referring to FIG. 27, a portable breast pumping positioning system 2700 is illustrated. In this example, the milk collection system 1300 can be used with the portable breast pumping system positioning 2700 such that the clamp 1304 is attached to any flat surface 2702, such as a table, counter, desk, or other furniture, for example, as explained in more detail above with reference to FIG. 14. The portable breast pumping positioning system 2700 also includes an upper portable support 2704 with a headrest aperture 2706 for receiving a headrest post (not shown) (e.g., headrest post 138), and a cushion attachment mechanism 2708 (e.g., hook and loop fastener) on a front angled portion for attaching a chest cushion (not shown) (e.g., chest cushion 638) or other cushion. In this example, the upper portable support 2704 includes one or more threaded posts 2710 extending from a bottom portion and configured to receive a plate 2712 or other portion of a lower portable support 2714 that is coupled to a knob 2716.

In use, the knob 2716 can be rotated by a user to move the plate 2712 and lower portable support 2714 upward along the threaded posts 2710 and toward a bottom of the flat surface 2702 to thereby act as a clamp or vice to retain the upper portable support 2704 and lower portable support 2714 in position with the flat surface 2702 disposed between those components. Optionally, the at least a portion of the bottom of the upper portable support 2704 and/or the top of the lower portable support 2714 can include rubber or another type of friction and/or compressible material to facilitate a tight fit capable of supporting a portion of a user's weight on the front angled portion of the upper portable support 2704. In some examples, the upper portable support 2704 is configured to overhang the flat surface 2702 in use to provide a more comfortable access and use of the milk collection system 1300.

Having described the basic concept of the technology, it should be apparent to those skilled in the art that the foregoing detailed disclosure is intended to be presented by way of example only and is not limiting. Various alterations, improvements, and modifications will occur and are intended to those skilled in the art, though not expressly stated herein. These alterations, improvements, and modifications are intended to be suggested hereby, and are within the spirit and scope of the invention.

What is claimed is:

1. A breast pumping chair system, comprising:
a chair assembly comprising a seat frame coupled to a base frame, a first foot and a second foot coupled to opposing ends of the base frame, a seat support coupled to the seat frame toward a first end of the seat frame, and an angle adjustment mechanism configured to alter an angle of the seat frame with respect to the base frame;
a breast pumping assembly comprising:
a chest frame coupled to the seat frame toward a second end of the seat frame, a chest support coupled to the chest frame, a chest cushion coupled to the chest support and angled to engage a sternum of a user in a forward leaning position, and a headrest post removably attached to the chest frame and coupled to a headrest support coupled to a headrest cushion; and
a milk collection system comprising first and second bottle holders rotatably coupled via first and second holder posts, respectively, to opposing sides of the chest frame, wherein each of the first and second bottle holders is configured to retain a bottle in place at a desired angle of rotation and distance from the user;
a height adjustment mechanism configured to alter a height of the breast pumping assembly with respect to the chair assembly; and
a table rotatably coupled to one or more of the chair assembly or the breast pumping assembly via a table hinge of a table adjustment mechanism configured to facilitate open and closed positions in which the table is substantially parallel and perpendicular to, respectively, a plane shared by the first and second feet.

2. The breast pumping chair system of claim 1, wherein the table is shaped to prevent engagement with the first and second bottle holders when the table is moved from the open position to the closed position.

3. The breast pumping chair system of claim 2, wherein the table adjustment mechanism further comprises a puller, a lock, an external table bracket removably coupled to the chest frame and comprising a plurality of detents, and an internal table bracket rotatably coupled to the external table bracket, wherein the puller is configured to disengage the table from one of the detents when engaged by the user and the lock is coupled to the table hinge to allow full rotation of the table to the closed position when engaged by the user.

4. The breast pumping chair system of claim 1, further comprising first and second cushioned leg supports coupled to opposing sides of the base frame.

5. The breast pumping chair system of claim 1, wherein the headrest post is adjustable along its length to vary a height of the headrest post with respect to the chest frame and the headrest support is rotatable to adjust an angle of the headrest cushion coupled thereto with respect to the headrest post.

6. The breast pumping chair system of claim 1, wherein the height adjustment mechanism comprises an interface disposed proximate the chest frame and configured to engage a pneumatic linear actuator disposed within and coupled between the chest frame and the seat frame, wherein the pneumatic linear actuator is configured to move the chest frame upward within the base frame.

7. The breast pumping chair system of claim 1, wherein the angle adjustment mechanism comprises a crank handle coupled to a threaded angle adjustment post disposed proximate the seat frame, wherein rotation of the crank handle and the threaded angle adjustment post is configured to raise and lower the seat frame with respect to the base frame.

8. The breast pumping chair system of claim 1, further comprising a grab bar coupled to the chest support and first and second handles removably coupled to opposing ends of the grab bar.

9. The breast pumping chair system of claim 8, further comprising a collection system bracket removably coupled to the chest frame and first and second collection system posts coupled to opposing sides of the collection system bracket.

10. The breast pumping chair system of claim 1, wherein at least one of the first or second bottle holders further comprises an upper component coupled to the first or second holder post, a lower component, and a holder bar configured to be received by the upper and lower components and coupled to a holder button configured to facilitate vertical movement of the lower component with respect to the upper component and about the holder bar, wherein the upper and lower components comprise first and second straps, respectively, which are configured to retain a bottle in place within the at least one of the first or second bottle holders.

11. The breast pumping chair system of claim 1, further comprising a seatbelt system comprising a pelvic support attached to the chest support, a pelvic cushion attached to the pelvic support, first and second female attachment mechanisms coupled to opposing ends of the pelvic support, and first and second male attachment mechanisms attached to opposing ends of a webbing and configured to engage with the first and second female attachment mechanisms, respectively.

12. The breast pumping chair system of claim 1, further comprising an adjustable arm configured to attach at a first end to an accessory bracket for a tissue warming or vibration device and at a second end to the breast pumping chair system via an arm connection mechanism.
